(19) United States
(12) Patent Application Publication
(10) Pub. No.: US 2018/0179388 A1
(43) Pub. Date: Jun. 28, 2018

(54) METHODS OF ANTHOCYANIN EXTRACTION FROM COLORED CORN CULTIVARS

(71) Applicants: KRAFT FOODS GROUP BRANDS LLC, CHICAGO, IL (US); THE BOARD OF TRUSTEES OF THE UNIVERSITY OF ILLINOIS, URBANA, IL (US)

(72) Inventors: Qian Li, Newburgh, IN (US); Elvira Gonzalez de Mejia, Monticello, IL (US); Vijay Singh, Champaign, IL (US); Pavel Somavat, Urbana, IL (US); Megan West, Palatine, IL (US); Leslie West, Winnetka, IL (US); Pat Donahue, Libertyville, IL (US)

(73) Assignees: KRAFT FOODS GROUP BRANDS LLC, CHICAGO, IL (US); THE BOARD OF TRUSTEES OF THE UNIVERSITY OF ILLINOIS, URBANA, IL (US)

(21) Appl. No.: 15/739,528

(22) PCT Filed: Jul. 22, 2016
(86) PCT No.: PCT/US16/43496

§ 371 Date: Dec. 22, 2017

Related U.S. Application Data
(60) Provisional application No. 62/195,834, filed on Jul. 23, 2015.

(51) Int. Cl. C09B 61/00; A01H 5/10; A61K 36/899; C07H 3/00; C07D 311/04

(52) U.S. Cl. CPC C09B 61/00; A01H 5/10; C07D 311/04; C07H 3/00; A61K 36/899

(57) ABSTRACT

Methods of extracting anthocyanins from corn kernels are disclosed. In embodiments, the methods may include fractionating the corn kernels into their constituent component parts. After fractionating, the pericarp fiber may be separated from the constituent component parts of the corn kernels. The pericarp fiber may be steeped in an aqueous solution to extract anthocyanins from the pericarp fiber. After steeping, the aqueous solution may contain greater than about 40% by weight of total extractable anthocyanins present in the corn kernels prior to fractionating.

### CROSS-REFERENCE TO RELATED APPLICATIONS

The present application claims priority to U.S. Provisional Patent Application Ser. No. 62/195,834, filed Jul. 23, 2015, and entitled “Methods of Anthocyanin Extraction from Colored Corn Cultivars,” the entirety of which is incorporated by referenced herein.

### FIELD

The present specification generally relates to methods for extracting anthocyanins from plants, and more particularly, to methods for extracting anthocyanins from the pericarp fiber of corn kernels.

### TECHNICAL BACKGROUND

Anthocyanins are water soluble vacuolar pigments belonging to the flavonoid group of phytochemicals found in plant tissue that contribute to the color of the plant tissue. Anthocyanins may present health benefits to individuals that consume anthocyanin-containing foods. For example, anthocyanins may play a key role in scavenging free radicals, which may be used in preventing or treating chronic degenerative diseases such as atherosclerosis, aging, diabetes, hypertension, inflammation, and cancer. As such, when extracted from the plant tissue, anthocyanins can be used for many purposes, including, but not limited to, medicine, health supplements, and natural food colorants.

Because of the natural origin and health benefits of anthocyanins, anthocyanin extraction from vegetables and fruits, such as berries and grapes, is of interest. However, the cost and yield of anthocyanin extraction by conventional techniques are unsatisfactory. Corn is the primary U.S. feed grain. However, anthocyanins present in purple or other colored corn co-products are under-utilized.

Accordingly, a need exists for efficient methods of extracting anthocyanins from plant tissues, such as from the tissues of corn kernels, while preserving the balance of the plant tissues for other purposes.

### SUMMARY

According to one embodiment, a method of extracting anthocyanins from corn kernels may include fractionating the corn kernels into their constituent component parts. After fractionating, the pericarp fiber may be separated from the constituent component parts of the corn kernels. The pericarp fiber may be steeped in an aqueous solution to extract anthocyanins from the pericarp fiber. After steeping, the aqueous solution may contain greater than about 40-70% by weight of total extractable anthocyanins present in the corn kernels prior to fractionating.

According to another embodiment, a method of extracting anthocyanins from corn kernels may include fractionating whole corn kernels with a degermination mill into a first fraction comprising grits and a second fraction comprising germ, pericarp fiber and ground corn. The pericarp fiber of the corn kernels may include greater than about 40-70% by weight of total extractable anthocyanins present in the corn kernels. Thereafter, the pericarp fiber is ground in a roller mill to obtain a ground second fraction. The ground second fraction is then passed through a sieve to separate the pericarp fiber from the germ. The recovered pericarp fiber is steeped in an aqueous solution to extract anthocyanins from the pericarp fiber. Thereafter, the anthocyanins may be isolated from the aqueous solution.

Additional features and advantages of the methods for anthocyanin extraction described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 schematically depicts a cross section of a corn kernel showing various constituent component parts of the plant tissue;

FIG. 2 schematically depicts the backbone structure of anthocyanin and the structure of six of the most common anthocyanins;

FIG. 3 is a flow diagram of a method for extracting anthocyanins from the pericarp fiber of corn kernels, according to one or more embodiments shown and described herein;

FIG. 4 schematically depicts a conventional dry grinding process;

FIG. 5 schematically depicts an enzymatic dry grinding process which may be used for separating pericarp fiber from the remaining constituent component parts of the corn kernel according to one or more embodiments shown and described herein;

FIG. 6 schematically depicts a dry fractionation process which may be used for separating pericarp fiber from the remaining constituent component parts of the corn kernel according to one or more embodiments shown and described herein;

FIG. 7 is a flow diagram of a conventional dry milling process which may be used for separating pericarp fiber from the remaining constituent component parts of the corn kernel according to one or more embodiments shown and described herein;

FIG. 8 is a flow diagram of a conventional wet milling process which may be used for separating pericarp fiber from the remaining constituent component parts of the corn kernel according to one or more embodiments shown and described herein;

FIG. 9 is a flow diagram of corn anthocyanin extractions of co-products obtained from wet-milled corn kernels;

FIG. 10A is a comparison of the amount of anthocyanins extracted from the pericarp steeping liquid co-products and further extractions from the remaining dried pericarp with 2% formic acid where anthocyanin concentrations are represented as mg of cyanindin-3-glucoside equivalents per g of dry pericarp, values followed by different letter(s) are statistically different from each other (p<0.05), values are means±SEM with significantly different letters (n=3, p<0.05), non-capital letters represent total extractable anthocyanin concentrations of steeping liquid and remaining dried pericarp in each product; and capital letters represent total extractable anthocyanin concentrations of steeping liquid in water, water+SO₂, water+lactic acid, and water+SO₂+lactic acid;

FIG. 10B depicts anthocyanin concentrations in Maiz Morado whole kernel from first to fifth extractions wherein anthocyanin concentrations are represented as mg of cyanindin-3-glucoside equivalent per g of pericarp and values are reported as means±SEM with significantly different letters (n=3, p<0.5);

FIG. 11(A) graphically depicts the concentrations of the total polyphenols in pericarp fiber aqueous steeping solution co-products wherein two sequential extractions with 2% formic acid from pericarp were individually diluted and measured, polyphenol concentrations from pericarp are the addition of two sequential extractions, total polyphenols are represented as mg of gallic acid equivalents per gram of dry pericarp, values followed by different letter(s) are statistically different from each other (p<0.05), and values are means±SEM with significantly different letters (n=3, p<0.05);

FIG. 11(B) graphically depicts the concentrations of the total flavonoids in pericarp fiber aqueous steeping solution co-products wherein two sequential extractions with 2% formic acid from pericarp were individually diluted and measured, flavonoid concentrations from pericarp are the addition of two sequential extractions, total flavonoids are represented as mg rutin equivalents per gram of dry pericarp, values followed by different letter(s) are statistically different from each other (p<0.05), and values are means±SEM with significantly different letters (n=3, p<0.05);

FIG. 11(C) graphically depicts the concentrations of the total tannins in pericarp fiber aqueous steeping solution co-products wherein two sequential extractions with 2% formic acid from pericarp were individually diluted and measured, tannins concentrations from pericarp are the addition of two sequential extractions, total tannins are represented as mg catechin equivalents per gram of dry pericarp, values followed by different letter(s) are statistically different from each other (p<0.05), and values are means±SEM with significantly different letters (n=3, p<0.05);

FIG. 12(A) graphically depicts HPLC chromatograms of corn pericarp aqueous steeping water co-products with only water at 520 nm;

FIG. 12(B) graphically depicts HPLC chromatograms of corn pericarp aqueous steeping water co-products with water plus SO₂ at 520 nm;

FIG. 12(C) graphically depicts HPLC chromatograms of corn pericarp aqueous steeping water co-products with water plus lactic acid at 520 nm;

FIG. 12(D) graphically depicts HPLC chromatograms of corn pericarp aqueous steeping water co-products with water plus SO₂ and lactic acid at 520 nm;

FIG. 13(A) graphically depicts HPLC chromatograms of corn pericarp aqueous steeping solution co-products with only water at 280 nm;

FIG. 13(B) graphically depicts HPLC chromatograms of corn pericarp aqueous steeping solution co-products with water plus SO₂ at 280 nm;

FIG. 13(C) graphically depicts HPLC chromatograms of corn pericarp aqueous steeping solution co-products with water plus lactic acid at 280 nm;

FIG. 13(D) graphically depicts HPLC chromatograms of corn pericarp aqueous steeping solution co-products with water plus SO₂ and lactic acid at 280 nm;

FIG. 14(A) graphically depicts HPLC chromatograms of corn pericarp aqueous steeping solution co-products with only water at 320 nm;

FIG. 14(B) graphically depicts HPLC chromatograms of corn pericarp aqueous steeping solution co-products with water plus SO₂ at 320 nm;

FIG. 14(C) graphically depicts HPLC chromatograms of corn pericarp aqueous steeping solution co-products with water plus lactic acid at 320 nm;

FIG. 14(D) graphically depicts HPLC chromatograms of corn pericarp aqueous steeping solution co-products with water plus SO₂ and lactic acid at 320 nm;

FIG. 15(A) graphically depicts HPLC chromatograms of corn pericarp aqueous steeping solution co-products with only water at 360 nm;

FIG. 15(B) graphically depicts HPLC chromatograms of corn pericarp aqueous steeping solution co-products with water plus SO₂ at 360 nm;

FIG. 15(C) graphically depicts HPLC chromatograms of corn pericarp aqueous steeping solution co-products with water plus lactic acid at 360 nm;

FIG. 15(D) graphically depicts HPLC chromatograms of corn pericarp aqueous steeping solution co-products with water plus SO₂ and lactic acid at 360 nm;

FIG. 16(A) graphically depicts the percentages (%) of anthocyanin distribution in pericarp aqueous steeping solution co-products as determined by HPLC at 520 nm in treatments with only water;

FIG. 16(B) graphically depicts the percentages (%) of anthocyanin distribution in pericarp aqueous steeping solution co-products as determined by HPLC at 520 nm in treatments with water plus SO₂;

FIG. 16(C) graphically depicts the percentages (%) of anthocyanin distribution in pericarp aqueous steeping solution co-products as determined by HPLC at 520 nm in treatments with water plus lactic acid; and

FIG. 16(D) graphically depicts the percentages (%) of anthocyanin distribution in pericarp aqueous steeping solution co-products as determined by HPLC at 520 nm in treatments with water plus SO₂ and lactic acid.

### DETAILED DESCRIPTION

Reference will now be made in detail to embodiments of methods for extracting anthocyanins from the pericarp fiber of corn kernels, examples of which are illustrated in the accompanying drawings. Whenever possible, the same reference numerals will be used throughout the drawing to refer to the same or like parts.

According to one embodiment, a method of extracting anthocyanins from the pericarp fiber of corn kernels may include fractionating the corn kernels into their constituent component parts. After fractionating, the pericarp fiber may be separated from the remaining constituent component parts of the corn kernels. The pericarp fiber may be steeped in an aqueous solution to extract anthocyanins from the pericarp fiber. After steeping, the aqueous solution may contain greater than about 40-70% by weight of total extractable anthocyanins present in the corn kernels prior to fractionating. Various embodiments of methods for extracting anthocyanins from the pericarp fiber of corn kernels will be described herein with specific reference to the appended drawings.

Specific and preferred values disclosed for components, ingredients, additives, temperatures, times, and like aspects, and ranges thereof, are for illustration only. They do not exclude other defined values or other values within defined ranges. The compositions, apparatuses, and methods of the disclosure include those having any value or combination of the values, specific values, or ranges thereof described herein.

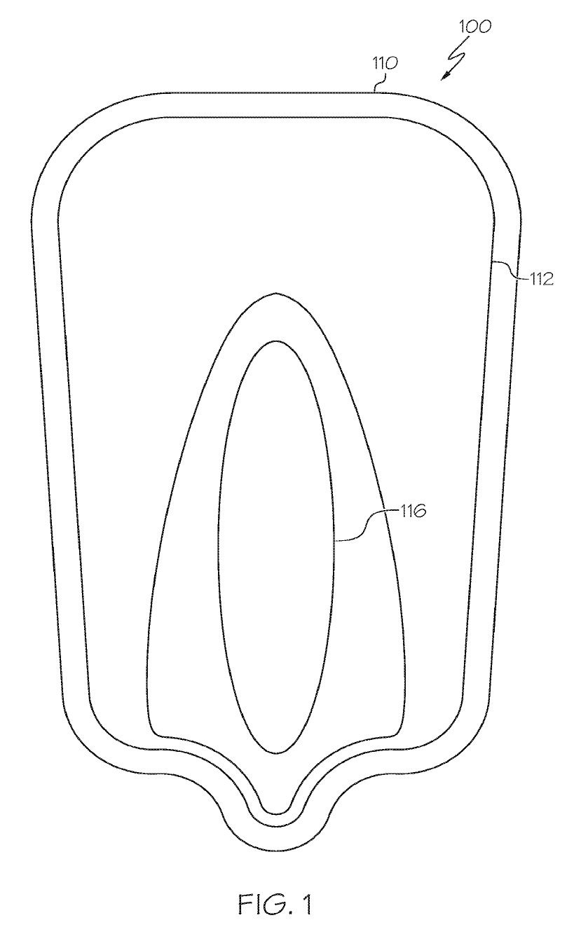

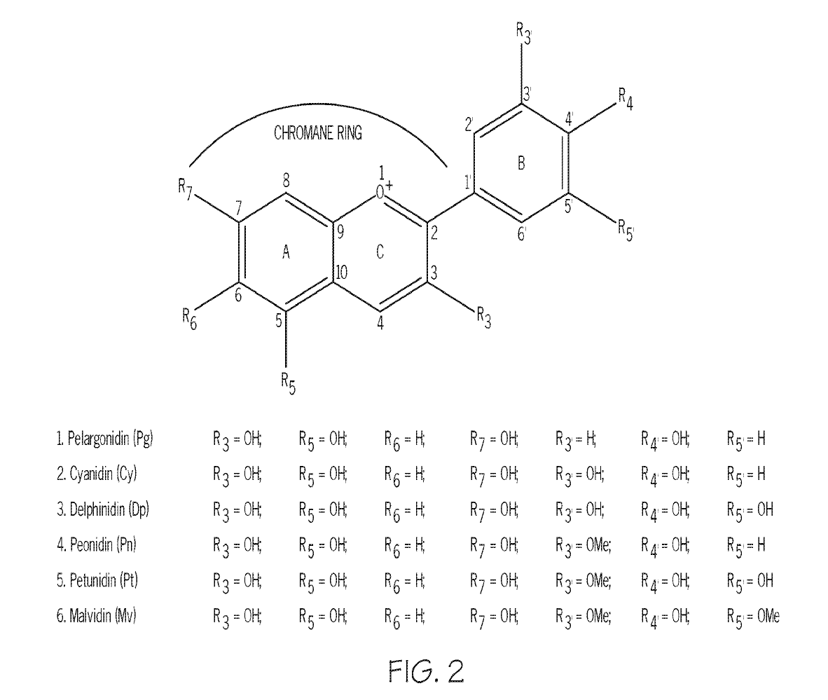

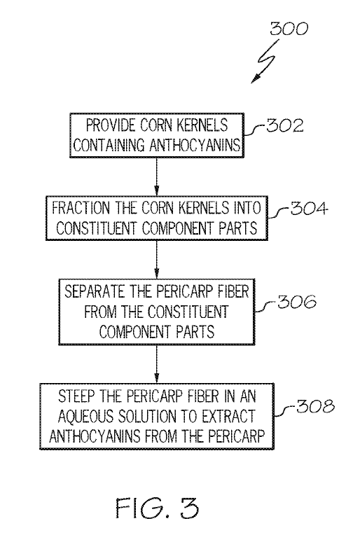

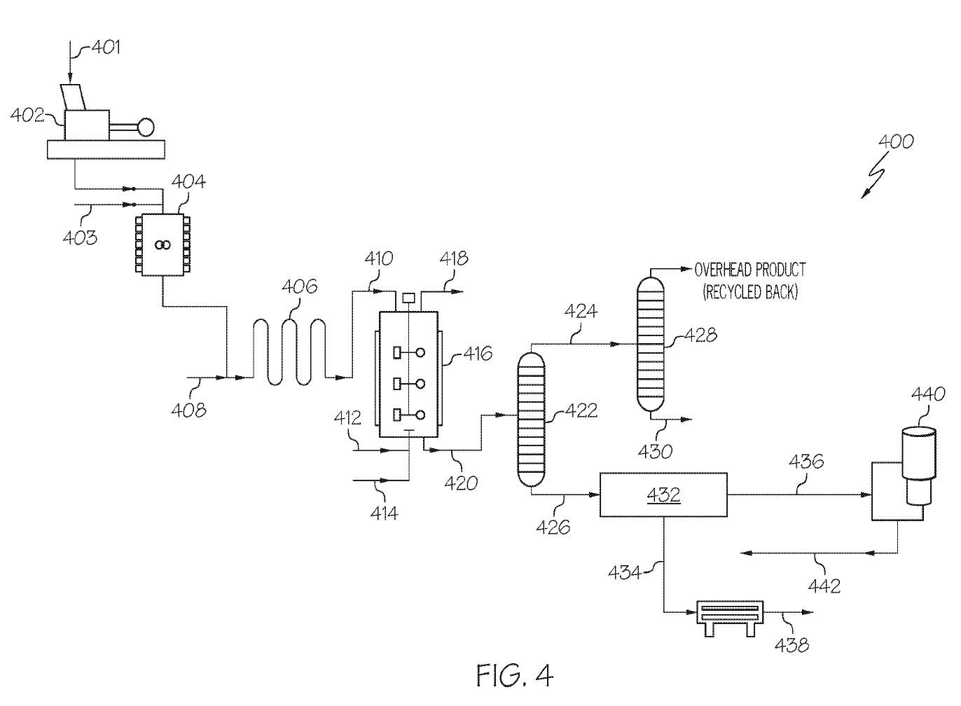

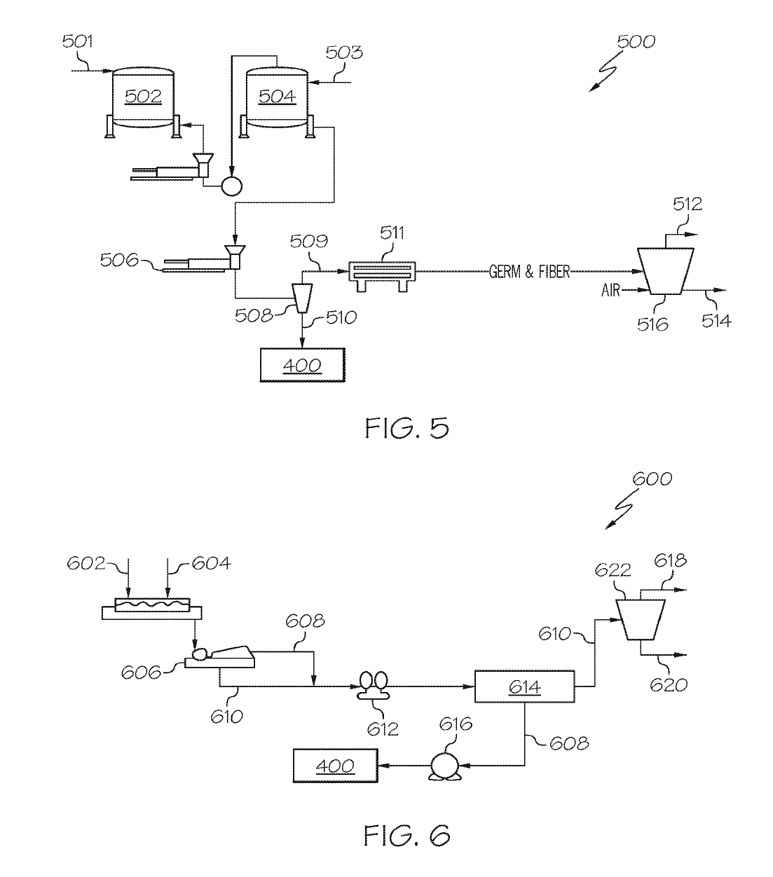

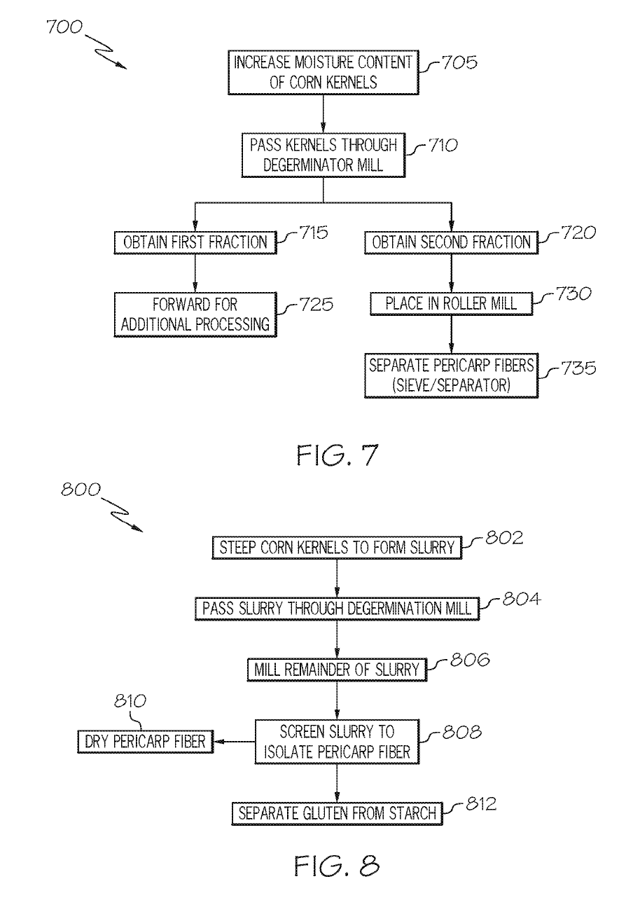

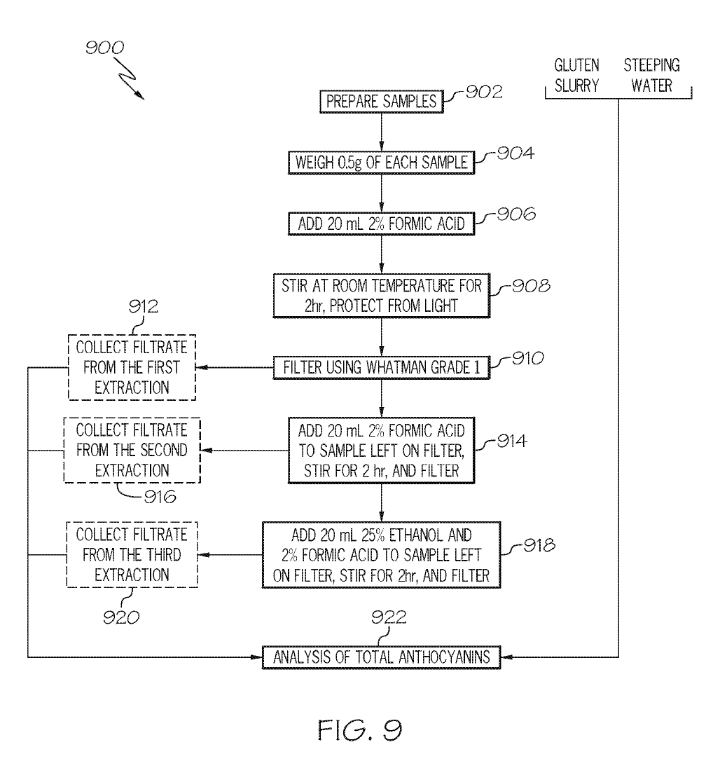

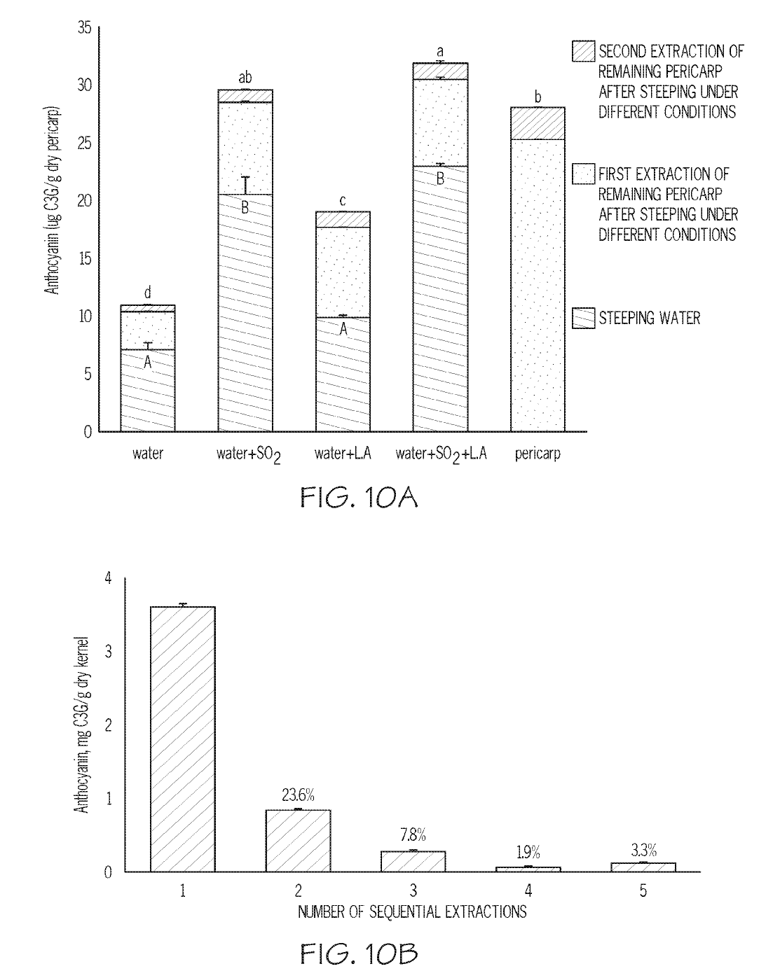

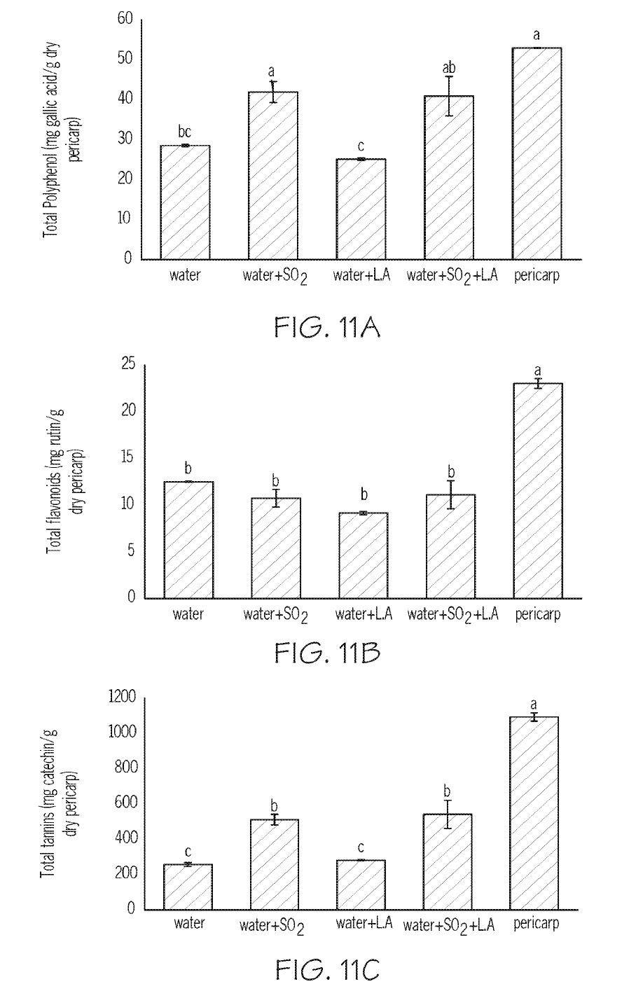

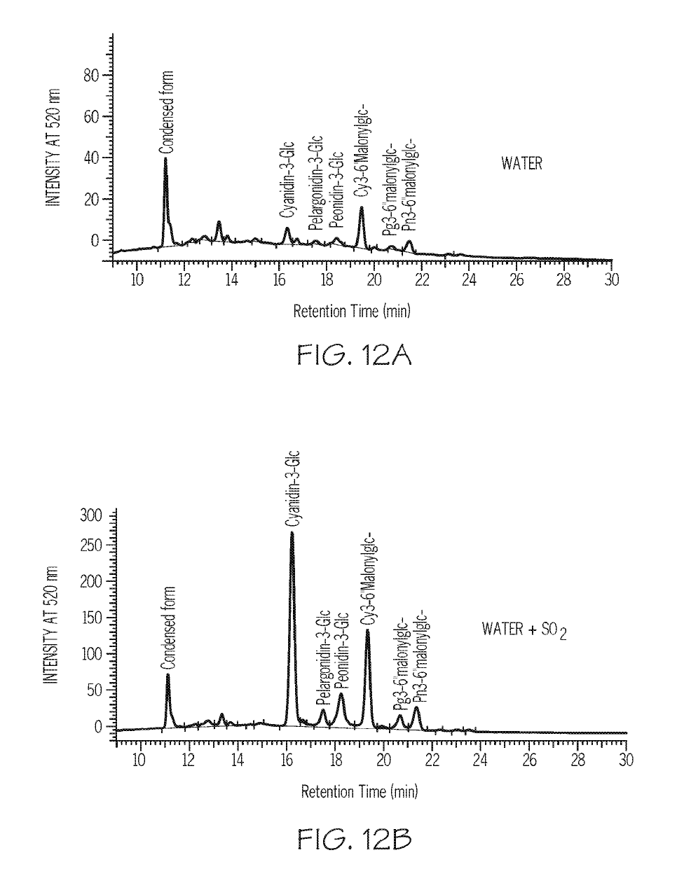

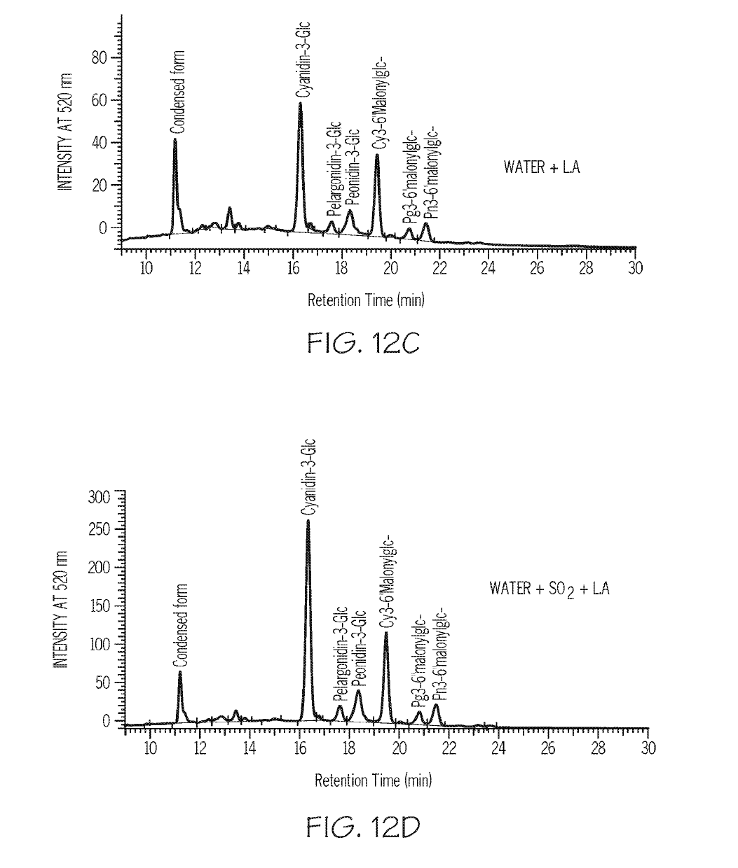

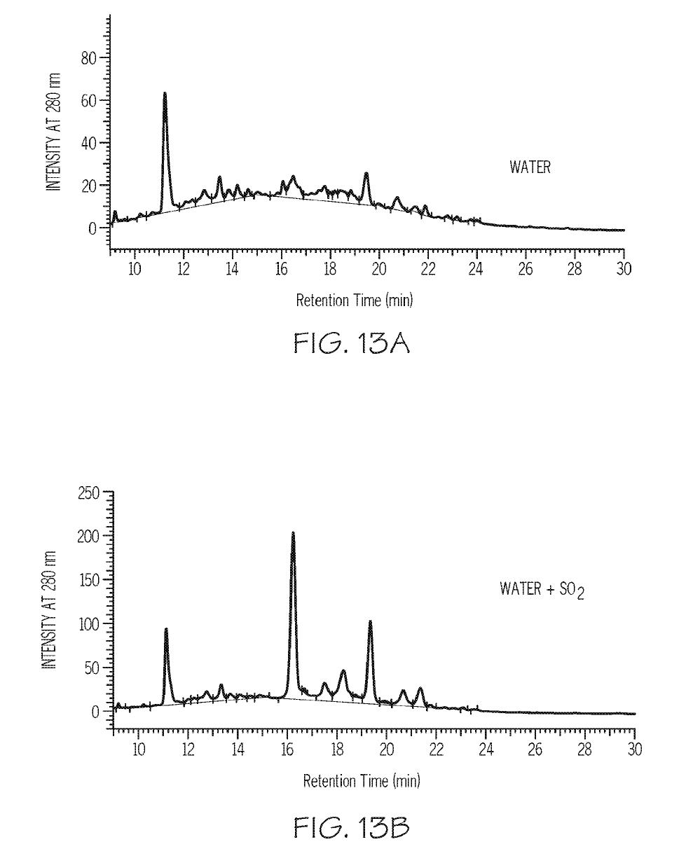

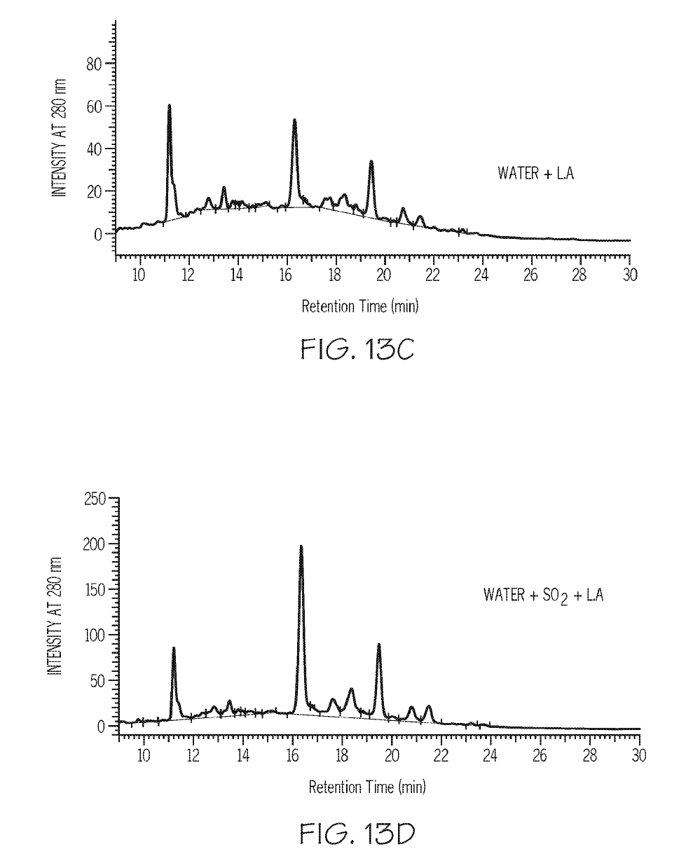

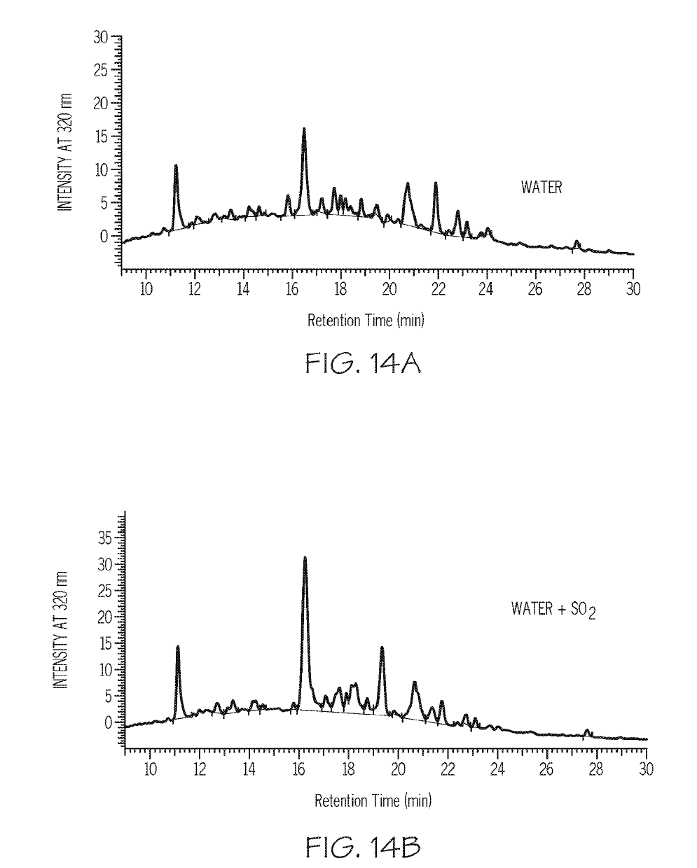

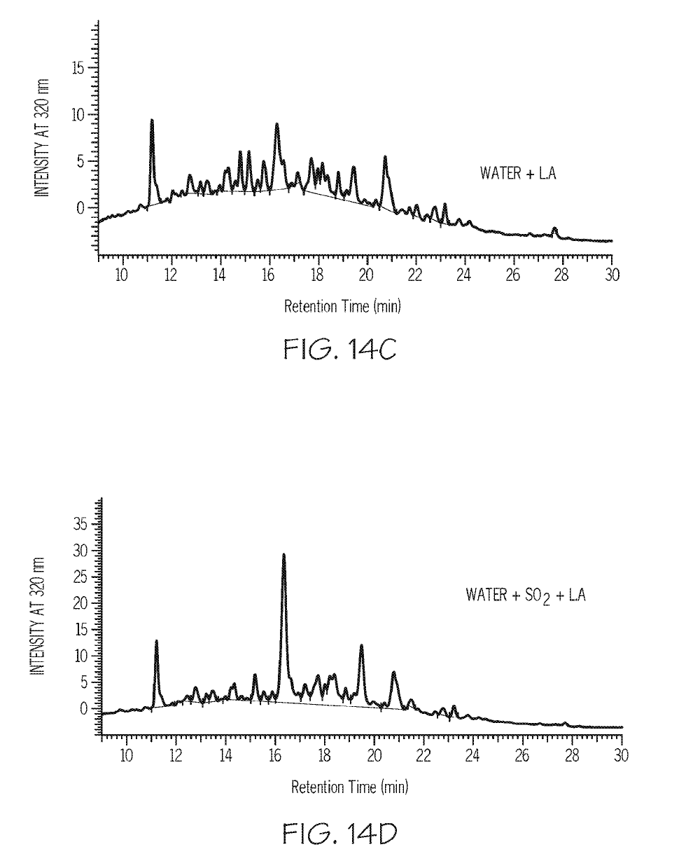

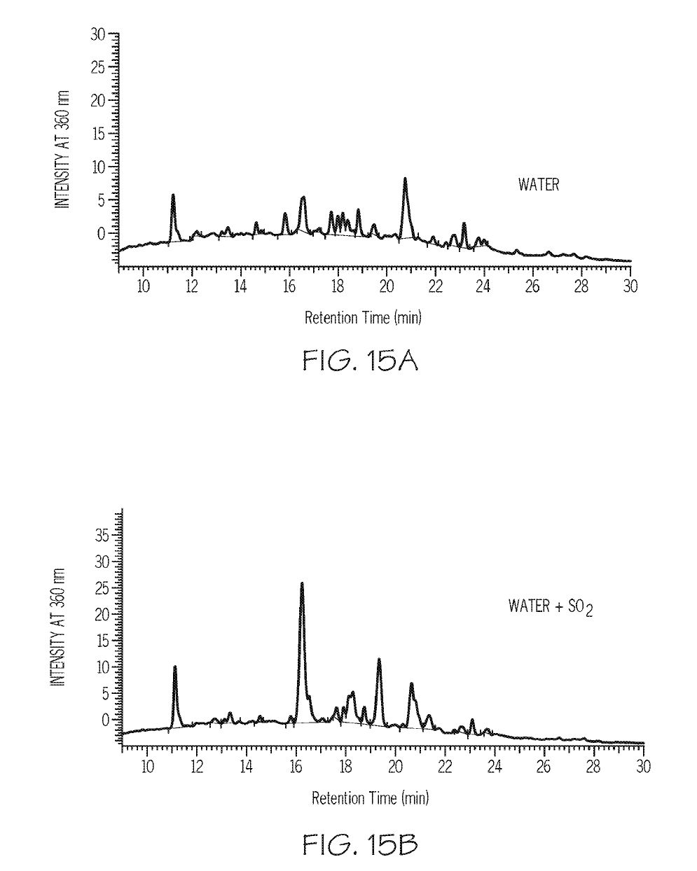

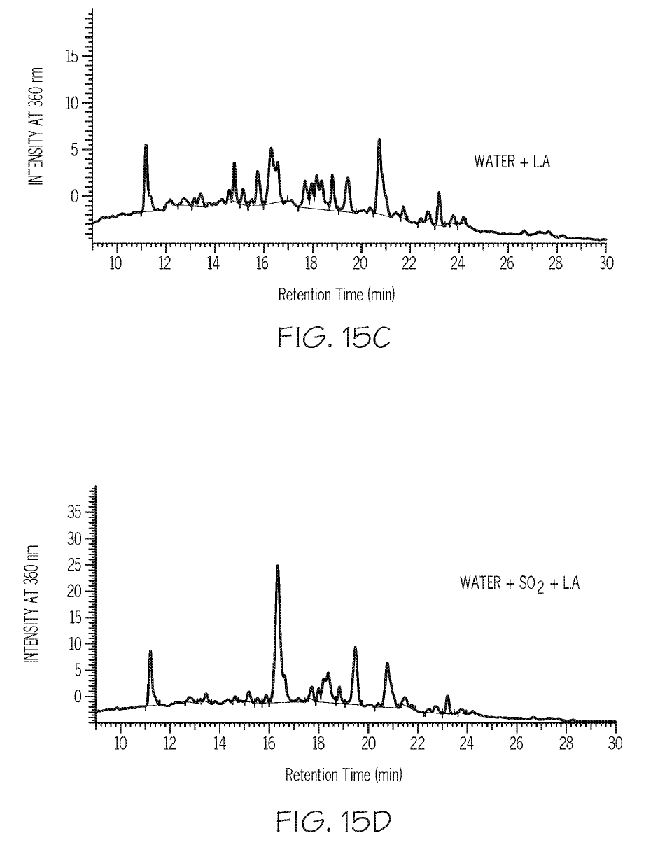

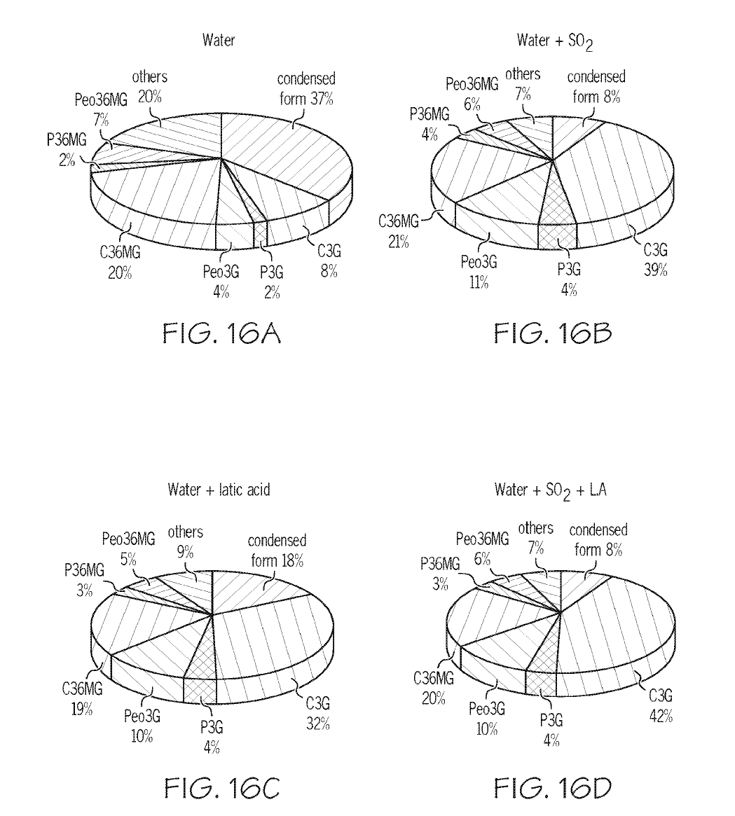

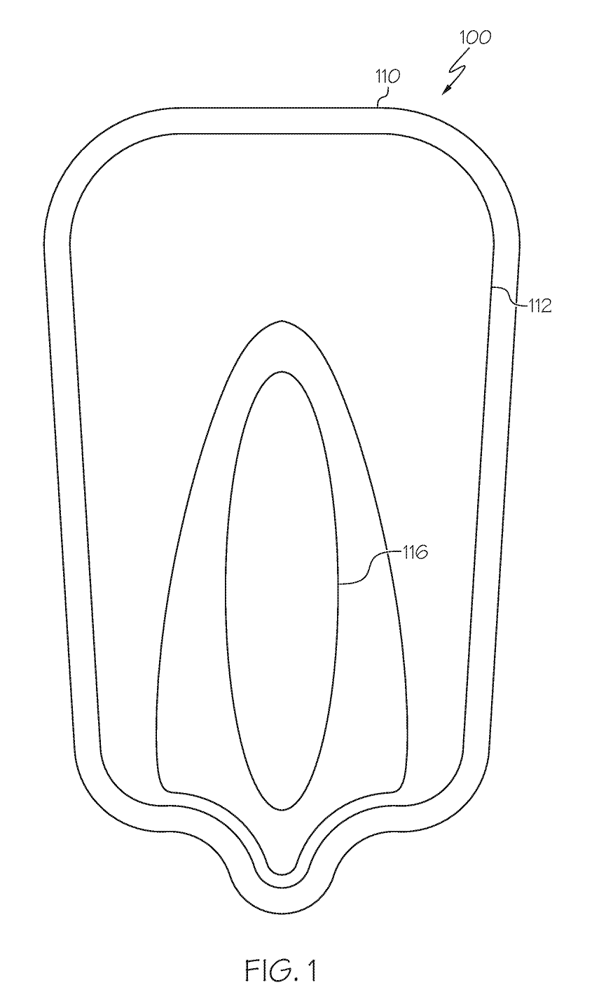

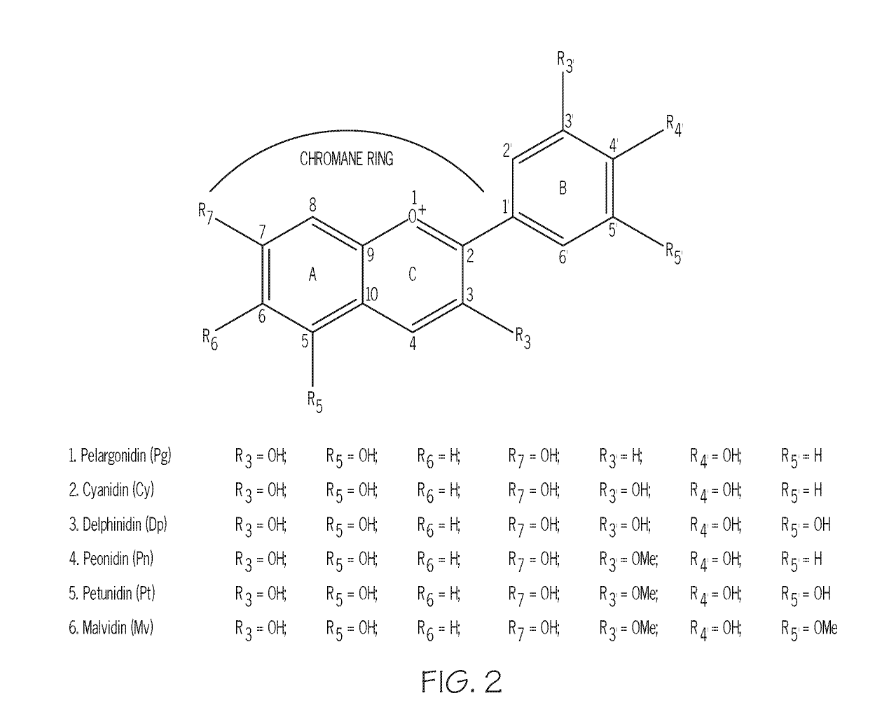

Anthocyanins occur naturally in the tissues of fruits and vegetables including in the tissues of corn (Zea mays). By way of example, FIG. 1 schematically depicts a cross section of a corn kernel 100 showing various constituent parts of the plant tissue including the pericarp fiber 110, the endosperm 112, and the germ 116. Structurally, the anthocyanins in fruits and vegetables are substituted glycosides and acylglycosides of 2-phenylbenzopyrilium salts (anthocyanidins). As depicted in FIG. 2, the basic structure of anthocyanidins includes a chromane ring (ring A and ring C) bearing a second aromatic ring (ring B) in position 2. The various anthocyanidins differ in number and position of the hydroxyl and/or methyl ether groups attached on 3, 5, 6, 7, 3′, 4′ and/or 5′ positions. Six of the most common structures are shown in FIG. 2, which include Pelargonidin (Pg) Cyanidin (Cy), Delphinidin (Dp), Peonidin (Pn), Petunidin (Pt), and Malvidin (Mv).

Referring again to FIG. 1, on a dry basis, the corn kernel 100 consists of approximately 10% by weight pericarp fiber, approximately 10% by weight germ, and approximately 80% by weight endosperm. The endosperm may be further subdivided into smaller fractions including large grits (˜22% by weight of the corn kernel), small grits (˜23% by weight of the corn kernel), and fines (˜35% by weight of the corn kernel). While anthocyanins may be present throughout the tissues of the corn kernel 100, in some cultivars, such as Maiz Morado, it has been determined that the greatest concentrations of anthocyanins occur in the pericarp fiber 110. More particularly, for Maiz Morado, it has been found that the pericarp fiber 110 of the corn kernel 100 contains greater than 65% of the extractable anthocyanins in the corn kernel 100 while the germ 116 contains less than 5% of the extractable anthocyanins in the corn kernel 100. The remaining extractable anthocyanins (˜30%) are resident in the endosperm 112 of the corn kernel 100 with ˜9% in the large grits, ˜12% in the small grits, and ˜23% in the fine grits. Based on these findings, greater than 65% of the extractable anthocyanins in the corn kernel 100 are present in the portion (i.e., the pericarp) of the corn kernel which accounts for only 10% or less of the weight of the corn kernel. Accordingly, in the embodiments described herein, anthocyanins are extracted only from the pericarp fiber of the corn kernel, increasing process efficiency and anthocyanin yield and decreasing waste as the remaining portions of the corn kernel (germ, grits) may be utilized for other purposes including, without limitation, ethanol production, supplements, cosmeceuticals, cosmetics, industrial products, food products for human consumption, non-human animal feed and the like.

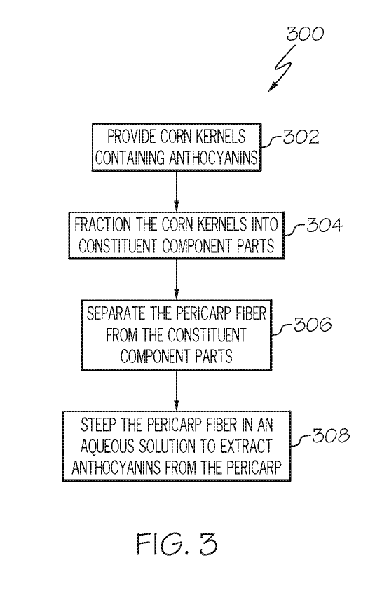

Referring to FIG. 3, a flow diagram of one embodiment of method 300 for extracting anthocyanins from the pericarp fiber of corn kernels is depicted. The method 300 may include an initial step 302 of providing corn kernels containing anthocyanins. In embodiments, the corn kernels provided in step 302 are selected from a variety of corn having a relatively high concentration of anthocyanins in the pericarp material. Such corn varieties may include, for example and without limitation, a “purple corn” or “blue corn” variety like Maiz Morado. In some embodiments, the variety of corn selected may have pericarp fiber which includes from about 45% by weight of the total extractable anthocyanins present in the whole corn kernel to about 80% by weight of the total extractable anthocyanins present in the whole corn kernel, including about 46% by weight, about 47% by weight, about 48% by weight, about 49% by weight, about 50% by weight, about 50% by weight, about 51% by weight, about 52% by weight, about 53% by weight, about 54% by weight, about 55% by weight, 56% by weight, about 57% by weight, about 58% by weight, about 59% by weight, about 60% by weight, about 61% by weight, about 62% by weight, about 63% by weight, about 64% by weight, about 65% by weight, about 66% by weight, about 67% by weight, about 68% by weight, about 69% by weight, about 70% by weight, or any value or range between any two of these values (including endpoints). In some embodiments, the pericarp fiber of the variety of corn selected may comprise from about 60% by weight to about 70% by weight of the total extractable anthocyanins present in the whole corn kernel. In some embodiments, the pericarp fiber of the variety of corn selected may comprise from about 65% by weight to about 70% by weight of the total extractable anthocyanins present in the whole corn kernel. In some embodiments, the pericarp fiber of the variety of corn selected may comprise greater than 65% by weight of the total extractable anthocyanins present in the whole corn kernel, greater than 67% by weight of the total extractable anthocyanins present in the whole corn kernel, or greater than 70% by weight of the total extractable anthocyanins present in the whole corn kernel. It should be understood that the particular amount of total extractable anthocyanins present in pericarp fiber may vary depending on the particular variety of corn selected.

Thereafter, in step 304, the corn kernels are fractioned into their constituent component parts (i.e., pericarp fiber, endosperm (large, small and fine grits), and germ) utilizing conventional corn processing techniques (e.g., wet milling, dry milling, dry grinding or modifications thereof). Fractionating the corn kernels into their constituent component parts allows the constituent component parts containing the greatest concentrations of anthocyanins (i.e., the pericarp fiber) to be separated and separately processed so as to minimize the amount of waste. That is, the remaining constituent component parts of the corn kernel (i.e., the germ and endosperm) can be used for additional processing, including, but not limited to, human food products, non-human animal feed, cosmeceuticals, cosmetics, supplements, industrial products, and other products, such as distiller's dried grains and solubles (DDGS), without undergoing processing for anthocyanin extraction. Exemplary conventional corn processing techniques suitable for fractionating corn kernels into their constituent component parts, such as wet milling, dry milling, and dry grinding, will now be described in further detail.

### Dry Grinding

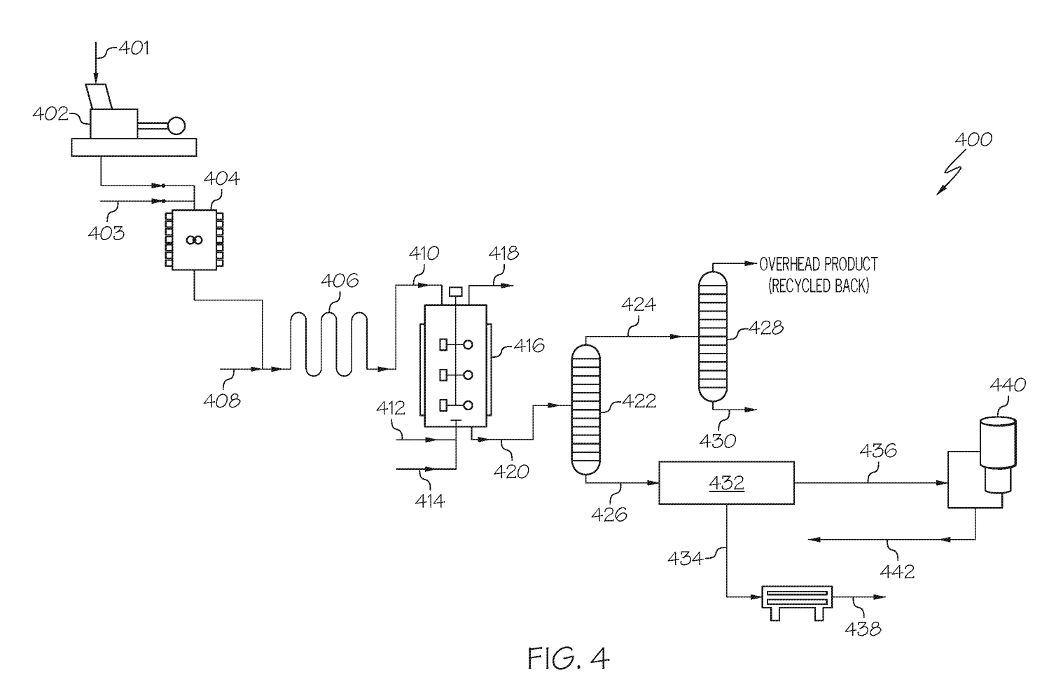

FIG. 4 depicts a conventional dry grinding process 400 suitable for fractionating corn kernels into their constituent component parts according to one or more embodiments. First, the whole corn kernels 401 are placed into a grinder 402, which grinds the whole corn kernels 401. The ground material is then mixed with water 403 to create a slurry 404 and the slurry 404 is passed into one or more jet-cookers 406. The jet-cookers 406 liquefaction the slurry by injecting steam into the slurry to cook it at temperatures in excess of 100° C. The liquefaction process breaks down the starch in the endosperm. Enzymes 408, such as alpha-amylase for example, are added to the slurry 404 and the added enzymes break down the starch copolymer. The resultant product may be referred to as a mash 410.

The mash 410 is cooled to a temperature of approximately 30° C. and yeast 412 and glucoamylase enzyme 414 is added to the mash 410 to facilitate the breakdown of starches into simple sugars in a process referred to as saccharification. The mixture of the mash 410, yeast 4112, and glucoamylase 414 is then fermented (which may take place simultaneously with saccharification) in a fermenting tank 416. The fermenting process yields CO₂ 418 and fermented corn mash (i.e., “beer”) 420.

The beer 420 is passed to a stripping/rectifying column 422 where solid portions 426 of the mash are separated from the liquid portions 424 of the beer. The liquid portions 424 of the beer are distilled in a distiller 428 to recover ethanol 430 while the solid portions 426 of the mash are centrifuged in a centrifuge 432 to separate the solid portions of the mash into wet grains 434 and thin sillage 436. The wet grains 434 are dried to produce distillers' dry grains (DDGS) 438 which are utilized as animal feed. The thin sillage 436 is directed into an evaporator 440 to recover water from the sillage 436, which is recycled into the process, and syrup 442 which is combined with the wet grains 434 and dried to produce the DDGS 438.

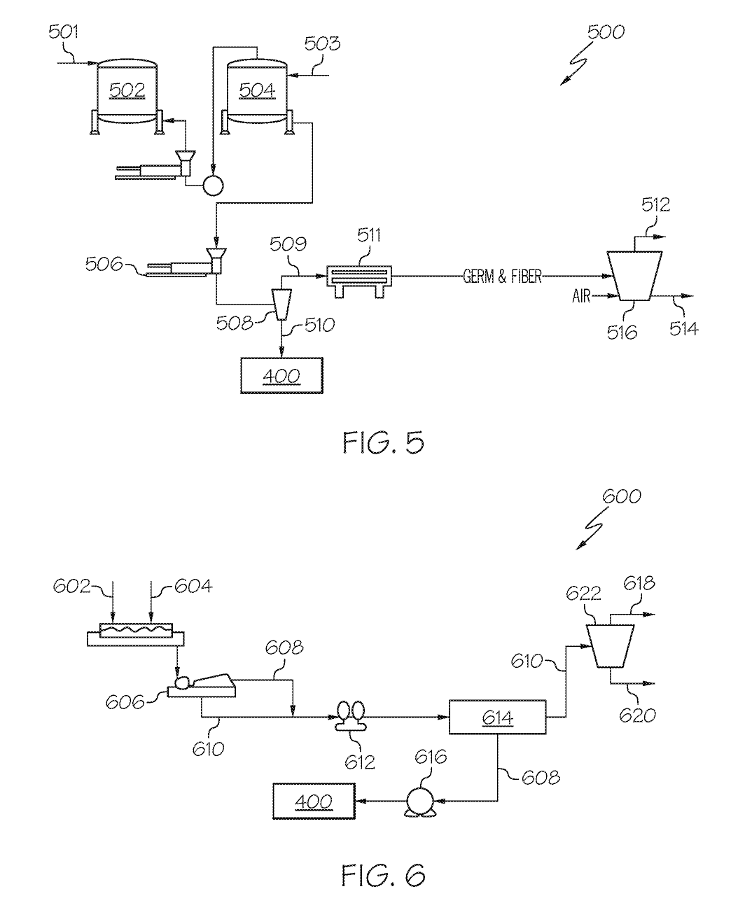

Referring now to FIG. 5, in embodiments, a modified dry grinding process 500 may be used to further recover pericarp fiber from the corn kernels. For example, FIG. 5 schematically depicts a modification of a conventional dry grind process referred to as an enzymatic dry grind corn process as described in Wang, P, Singh, V. Xu, L., Johnston, D. B., Rausch, K. D., and Tumbleson, M. E., 2005, Comparison of enzymatic (E-Mill) and conventional dry grind corn processes using a granular starch hydrolyzing enzyme, Cereal Chem. 82:734-738. In this process, the corn kernels 501 are initially soaked in water 502. Thereafter, the corn kernels are combined with enzymes 503, such as GSH and protease for example, and incubated in a holding tank 504. The mixture is fed to a degermination mill 506, such as a Bauer degermination mill, for grinding. The ground material is then fed to a separator 508 and the germ and pericarp fiber 509 are separated from the endosperm 510. The endosperm 510 is then further ground in a Bauer degermination mill, such as a Beall degermination mill, and the ground endosperm may be further processed into ethanol and DDGS, as described above.

After the germ and pericarp fiber 509 are separated from the endosperm 510, the germ and pericarp fiber 510 are dried in a germ and fiber dryer 511 and subsequently separated into pericarp fiber 512 and germ 514 portions. In embodiments, separation may be accomplished using an aspirator 516, as shown in FIG. 5, or, alternatively using sieves or other conventional separation techniques for separating particulate material. The separated pericarp fiber 512 may be used for anthocyanin recovery, as described further herein, and the germ 514 may be further processed to recover corn oil form the germ with the balance of the germ being used to produce animal feed.

Referring now to FIG. 6, another modified dry grinding process 600 which may be used to recover pericarp fiber from the corn kernels is schematically depicted. This modified dry grinding process may be referred to as 3D process as described in Murthy, G. S., Singh, V., Johnston, D. B., Rausch, K. D., and Tumbleson, M. E., 2006, Evaluation and strategies to improve fermentation characteristics of modified dry grind corn processes, Cereal Chem. 83:455-459. In this process, the moisture content of the corn kernels is initially increased by exposing the corn kernels 602 to steam 604. Thereafter, the corn kernels are fed to a degermination mill 606, such as a Beall degerminator, where the corn kernels are separated into tails 608 (large, small, and fine endosperm “grits”) and throughs 610 (pericarp fiber and germ). The tails 608 and throughs 610 are passed through a roller mill 612 for further grinding. The ground material is then processed with a sifter 614 to separate the tails from the throughs. The tails are further ground with a hammer mill 616 and the ground tails may be further processed into ethanol and DDGS, as described above.

After the throughs 610 are separated from the endosperm grits, the germ and pericarp fiber are separated into pericarp fiber 618 and germ portions 620. In embodiments, separation may be accomplished using an aspirator 622, as shown in FIG. 6, or, alternatively using sieves or other conventional separation techniques for separating particulate material. The separated pericarp fiber 618 may be used for anthocyanin recovery, as described further herein, and the germ 620 may further processed to recover corn oil form the germ with the balance of the germ used to produce animal feed.

### Dry Milling

In alternative embodiments, dry milling may be used to separate the pericarp fiber from the remaining constituent component parts of the corn kernel. In general, the dry milling process involves separating the various constituent component parts of the corn kernel via one or more grinding operations such that the various components can be further separated and/or processed depending on their intended use. Such a dry milling process may be effective in separating a pericarp fiber of the corn kernel from the remaining portions of the corn kernel, such as, for example, large grits, small grits, fines, and germ.

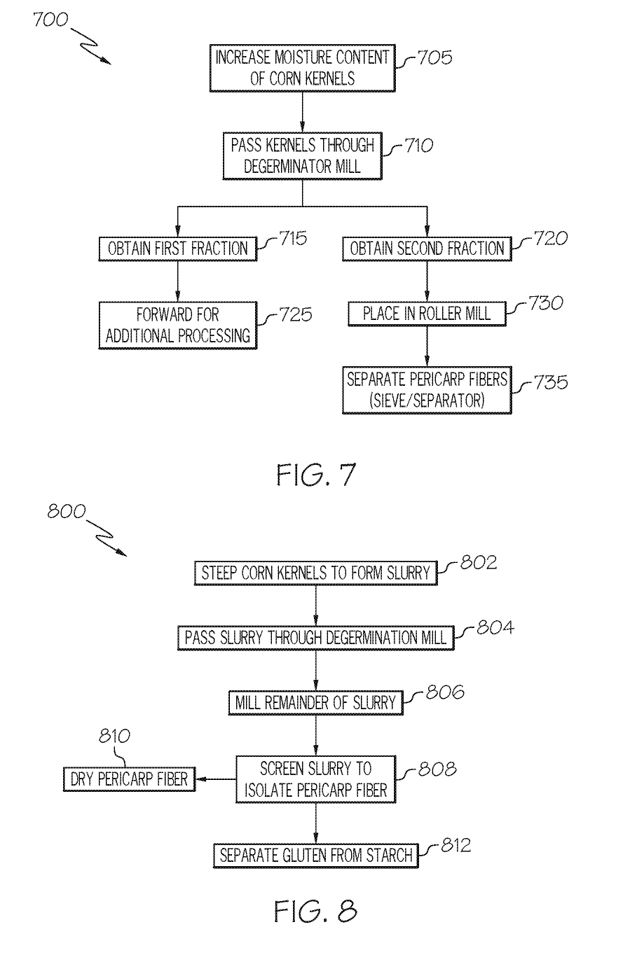

One conventional dry milling process is referred to as the “tempering-degerming process.” A flow diagram of the process is depicted in FIG. 7. While FIG. 7 sets forth specific steps of one exemplary dry milling process, it should be understood that the dry milling process described herein may be modified with additional steps that are generally recognized as dry milling steps without departing from the scope of the present disclosure.

The dry milling process 700 may include the initial step 705 of increasing the moisture content of the corn kernels by, for example, soaking the corn kernels or exposing the corn kernels to steam. The moisture content may generally be increased relative to the standard moisture content of the corn kernels prior to step 705. In some embodiments, the moisture content of the corn kernels may be increased such that the corn kernels comprise greater than about 15% water by weight or even greater than about 20% water by weight. In some embodiments, the moisture content of the corn kernels may be increased to about 21% water by weight or even about 22% water by weight, or any value or range between any two of these values (including endpoints).

In step 710, the corn kernels may be passed through a degermination mill. One illustrative degermination mill is a Beall degerminator. Passing the corn kernels through the degermination mill separates the corn kernel into its various constituent component parts (e.g., fractions). A first fraction, referred to as a tails fraction, may generally include the endosperm portion of the corn kernel in three “grit sizes” (i.e., large, small, and fines). A second fraction, referred to as a throughs fraction, may generally include the pericarp fiber and the germ of the corn kernel. It should also be understood that the tails fraction and the throughs fraction may each, respectively, contain other parts of the corn kernel not specifically described herein. For example, in some embodiments, a small portion of the endosperm may be present in the second fraction, which may be removed via additional processing via the degermination mill. Thus, as a result of the separation by the degermination mill, the first fraction may be obtained in step 715 and the second fraction may be obtained in step 720.

In step 725, the first fraction may be forwarded for additional processing. Such additional processing may include, but is not limited to, passing the first fraction through one or more roller mills, passing the first fraction through one or more sieves, and/or passing the first fraction through one or more separators. The first fraction may also be further processed and used for the manufacture of supplements, cosmeceuticals, cosmetics, industrial products, food products for human consumption, and/or for non-human animal feed products.

The second fraction (the throughs) is further processed to isolate the pericarp fiber from the germ and other portions of the second fraction. For example, in step 730, the second fraction may be placed in a roller mill for further grinding.

After the second fraction has been further ground in step 730, the various parts of the second fraction may be separated in step 735 to isolate the pericarp fiber from the second fraction. Such separation may be completed with one or more sieves, aspirators, and/or separators or other conventional separation techniques for separating particulate material. The separated pericarp fiber may be used for anthocyanin recovery, as described further herein, and the germ and other portions of the second fraction may be further processed to recover corn oil form the germ with the balance of the germ used to produce animal feed.

Once the pericarp fibers have been isolated from the second fraction, the remainder of the second fraction may optionally be forwarded for additional processing, similar to the additional processing of the first fraction, as described herein with respect to step 725.

### Wet Milling

In alternative embodiments, wet milling may be used to separate the pericarp and/or endosperm fiber from the remaining constituent component parts of the corn kernel. In general, the wet milling process involves separating the various constituent component parts of the corn kernel via steeping combined with one or more grinding/milling operations and screening operations such that the various components can be further separated and/or processed depending on their intended use. Such a wet milling process may be effective in separating a pericarp and/or endosperm fiber portion of the corn kernel from the remaining portions of the corn kernel.

A flow diagram of a conventional wet milling process 800 is depicted in FIG. 8. While FIG. 8 sets forth specific steps of one exemplary wet milling process, it should be understood that the wet milling process described herein may be modified with additional steps that are generally recognized as wet milling steps without departing from the scope of the present disclosure.

In a first step 802, the corn kernels are steeped in an aqueous solution to form a slurry. The steeping process may be performed at temperatures of approximately 50° C. for steeping times from about 20 hours to about 30 hours. The steeping process swells the corn kernels, softening and loosening the kernels and degrading the gluten bonds within the corn kernel, causing the release of starch into the steeping solution. In embodiments, SO₂ may be added to the solution to assist in the breakdown of the protein matrix within the kernels and increase the starch yield.

In step 804 the slurry is passed through a degermination mill to separate the germ from the remainder of the corn kernel. The separated germ may be further processed to recover corn oil form the germ with the balance of the germ used to produce animal feed.

In step 806, the remainder of the slurry is milled, such as by ball, disk, or impact milling or the like, to separate the starch and gluten from the pericarp fiber. Thereafter, in step 808, the slurry is screened to capture the pericarp fiber from the slurry. In step 810, the pericarp fiber recovered from the screen(s) is dried. The dried pericarp fiber may, thereafter, be used for anthocyanin recovery, as described further herein.

In step 812, the remaining slurry, which now consists of a starch-gluten suspension, is passed through a centrifuge where the gluten is separated from the starch. Thereafter, the gluten may be dried and used for the production of animal feed. The starch is then washed to remove any trace amounts of gluten. The starch may be dried to produce corn starch, or may be further processed to produce corn-based sweeteners, corn syrups, dextrose, and fructose.

Referring again to FIG. 3, after the corn kernels are fractioned into their constituent component parts, in step 306, the pericarp fiber is separated from the constituent component parts of the corn kernel, as described hereinabove, for further processing and the extraction of anthocyanins from the fiber. In some embodiments, such as where the corn kernels are dry milled or dry ground, the pericarp fiber may be separated from the remaining constituent component parts of the corn kernel by sieving or aspirator separation using, for example, a Multi-aspirator manufactured by Kice Industries. In alternative embodiments, the pericarp fiber may be separated from the remaining constituent component parts of the corn kernel by density separation using, for example, cyclone separators, dry density separation equipment available from, for example, Triple/S Dynamics, or wet density separation equipment.

In embodiments, after the pericarp fiber is separated from the constituent component parts of the corn kernel, the pericarp fiber may be, optionally, further ground to enhance the separation of anthocyanin-containing cellular material from non-anthocyanin-containing cellular material which further improves the efficiency of the subsequent anthocyanin extraction process. In embodiments, the pericarp fiber may be ground by, for example, roller milling or hammer milling. Further grinding of the pericarp fiber produces ground pericarp fiber containing two primary constituent components: anthocyanin-containing cellular material and non-anthocyanin-containing cellular material.

After grinding, the ground pericarp material may be further separated to isolate anthocyanin-containing cellular material from non-anthocyanin-containing cellular material. Separation may be completed via sieving and/or via density separation of the ground pericarp material. Thus, the material may be separated by sieving only, by density separation only, or by a combination of sieving and density separation. In embodiments, the mesh size of the sieve may be less than 425 microns (No. 40 sieve), such as 250 microns (No. 60 sieve), 180 microns (No. 80 sieve), or 125 microns (No. 120 sieve). Density separation, such as conventional wet density separation, may generally include placing the material in a liquid and processing the liquid through equipment that causes the material to separate based on density.

Referring again to FIG. 3, in step 308 the pericarp fiber (either in total or just the anthocyanin-containing cellular material portion) may be steeped in an aqueous solution such that the anthocyanins are extracted from the pericarp fiber into the aqueous solution. Since anthocyanins are highly soluble in water, steeping the pericarp fiber in an aqueous solution results in a highly efficient extraction of the anthocyanins from the pericarp fiber.

The pericarp fiber is steeped for a period of time sufficient to allow the anthocyanins to extract from the pericarp fiber. In some embodiments, the pericarp fiber may be steeped in the aqueous solution for a period of time from about 10 minutes to about 48 hours, including about 10 minutes, about 20 minutes, about 30 minutes, about 1 hour, about 2 hours, about 3 hours, about 4 hours, about 6 hours, about 8 hours, about 10 hours, about 12 hours, about 18 hours, about 24 hours, about 36 hours, about 48 hours, or any value or range between any two of these values (including endpoints). In some embodiments, the pericarp fiber may be steeped in the aqueous solution for about 24 hours.

In addition to a particular period of time, the pericarp fiber may also be steeped in the aqueous solution at a temperature sufficient to cause the anthocyanins to extract from the pericarp fiber. For example, the pericarp fiber may be steeped in the aqueous solution at a temperature from about 20° C. to about 95° C., including about 20° C., about 30° C., about 40° C., about 50° C., about 60° C., about 70° C., about 80° C., about 95° C., or any value or range between any two of these values (including endpoints). In some embodiments, the temperature may be about room temperature, or from about 20° C. to about 22° C., including about 20° C., about 20.5° C., about 21° C., about 21.5° C., about 22° C., or any value or range between any two of these values (including endpoints). In some embodiments, the temperature may be about 50° C.

In embodiments, the aqueous solution in which the pericarp fiber is steeped may comprise only water. In some embodiments, the water may be deionized water. In some embodiments, the aqueous solution may also contain at least one other component suitable for improving the efficacy of anthocyanin extraction from the pericarp. Such components may include, but are not limited to, a reducing compound, an organic acid or a combination thereof. For example, in some embodiments, the aqueous solution may contain water and at least one reducing compound. In other embodiments, the aqueous solution may contain water and at least one organic acid. In still other embodiments, the aqueous solution may contain water, at least one reducing compound, and at least one organic acid.

The reducing compound may be any reducing compound suitable for use in conjunction with the production of food products. In some embodiments the reducing compound may be a sulfite compound. Exemplary sulfite compounds suitable for use in the production of food products include, without limitation, sulfur dioxide, sodium sulfite, sodium bisulfite, sodium metabisulfite, potassium metabisulfite, potassium sulfite, calcium sulfite, calcium hydrogen sulfite, and potassium hydrogen sulfite.

When present in the aqueous solution, the reducing compound may be present in a concentration from about 5 parts per million to about 4000 parts per million, including, but not limited to, about 5 parts per million, about 10 parts per million, about 25 parts per million, about 50 parts per million, about 100 parts per million, about 200 parts per million, about 300 parts per million, about 400 parts per million, about 500 parts per million, about 600 parts per million, about 700 parts per million, about 800 parts per million, about 900 parts per million, about 1000 parts per million, about 2000 parts per million, about 3000 parts per million, about 4000 parts per million, or any value or range between any two of these values (including endpoints). In some embodiments, the reducing compound may be present in the aqueous solution at a concentration from about 1500 parts per million to about 2000 parts per million, including, but not limited to, about 1500 parts per million, about 1600 parts per million, about 1700 parts per million, about 1800 parts per million, about 1900 parts per million, about 2000 parts per million, or any value or range between any two of these values (including endpoints).

The organic acid may be any organic acid suitable for use in conjunction with the production of food products. In some embodiments, the organic acid may be lactic acid, a compound containing lactic acid, a derivative of lactic acid, and/or the like. In some embodiments, the organic acid may be selected from acetic acid, formic acid, propionic acid, butyric acid, valeric acid, caproic acid, oxalic acid, malic acid, citric acid, benzoic acid, and carbonic acid. In some embodiments, the organic acid may be a combination of two or more organic acids.

When present in the aqueous solution, the organic acid may be in the aqueous solution in a concentration from about 0.01% by weight to about 20% by weight of the aqueous solution, including, but not limited to, about 0.01% by weight, about 0.02% by weight, about 0.03% by weight, about 0.04% by weight, about 0.05% by weight, about 0.1% by weight, about 0.2% by weight, about 0.3% by weight, about 0.4% by weight, about 0.5% by weight, about 0.6% by weight, about 0.7% by weight, about 0.8% by weight, about 0.9% by weight, about 1% by weight, about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, or any value or range between any two of these values (including endpoints). In some embodiments, the organic acid may be present in the aqueous solution at a concentration from about 0.5% by weight to about 2% by weight, including about 0.5% by weight, about 1% by weight, about 1.5% by weight, about 2% by weight, or any value or range between any two of these values (including endpoints).

In some embodiments, following steeping, the aqueous solution may contain greater than about 40% by weight of total extractable anthocyanins present in the corn kernels prior to fractionating, such as when the steeping solution contains a reducing compound. For example, in some embodiments, such as embodiments employing Maiz Morado, after steeping, the aqueous solution contains greater than about 65% by weight of the total extractable anthocyanins present in the corn kernels prior to fractionating. In some other embodiments, after steeping, the aqueous solution contains greater than about 67% by weight of the total extractable anthocyanins present in the corn kernels prior to fractionating. In still other embodiments, after steeping, the aqueous solution contains greater than about 70% by weight of the total extractable anthocyanins present in the corn kernels prior to fractionating.

Steeping the pericarp fiber in an aqueous solution increases the efficiency of the anthocyanin extraction due to the high solubility of anthocyanin in water and increases the yield of anthocyanins from the corn kernels. In particular, it was found that steeping in an aqueous solution of water and a reducing compound such as SO₂ increased anthocyanin yields relative to an aqueous solution of only water by 200% and also increased anthocyanin yields relative to an aqueous solution of water and an organic acid, such as lactic acid. It was also found that steeping in an aqueous solution of water, a reducing compound such as SO₂, and an organic acid such as lactic acid not only increased anthocyanin yields relative to an aqueous solution of only water by more than 200%, but also yielded a steeping solution containing anthocyanins with better, more stable color.

Further, it has also been found that grinding the pericarp fiber after separating the pericarp fiber from the remainder of the constituent component parts of the corn kernel but before anthocyanin extraction may further increase the yield of anthocyanins from the corn kernel. Specifically, it has been found that grinding the pericarp fiber, either by ball milling or hammer milling, and sieving the ground pericarp fiber through a sieve having a mesh size less than 425 microns increased the yield of anthocyanin from the corn kernel. In addition, it was also found that ball milling the pericarp fiber followed by sieving the ground pericarp fiber through a sieve having a mesh size less than 425 microns increased the yield of anthocyanin from the corn kernel relative to hammer milling the pericarp fiber followed by sieving the ground pericarp fiber through a sieve having a mesh size less than 425 microns.

After the steeping step is completed, the pericarp fiber may be removed from the aqueous solution by, for example, filtering. Thereafter, the aqueous solution, now containing anthocyanins, may be processed to separate the anthocyanins from the aqueous solution. Suitable methods for separating the anthocyanins from the aqueous solution may include, for example and without limitation, column chromatography. In some embodiments, the aqueous solution containing the anthocyanins is combined with an extraction solution and passed through a filter to isolate the anthocyanins. The extraction solution may include at least one of an alcohol, an organic acid, and an enzyme, as will be described in greater detail in the examples below. The separated anthocyanins may be used for the production of various consumable products including, without limitation, medicines, health supplements, and natural food colorants.

### EXAMPLES

The embodiments described herein will be further clarified by the following examples.

### Example 1

Experiments were conducted to assess the yield of anthocyanins from whole corn kernels and from only the pericarp portion of the corn kernels using a corn variety containing high anthocyanins, specifically purple corn. The purple corn used in these experiments was the Maiz Morado corn cultivar purchased from Angelina's Gourmet (Swanson, Conn.). All chemicals were purchased from Sigma-Aldrich (St. Louis, Mo.) unless otherwise stated.

Sample Preparation and Anthocyanin Extraction

(1) Extraction of Anthocyanin From Wet-Milled Whole Corn Kernels

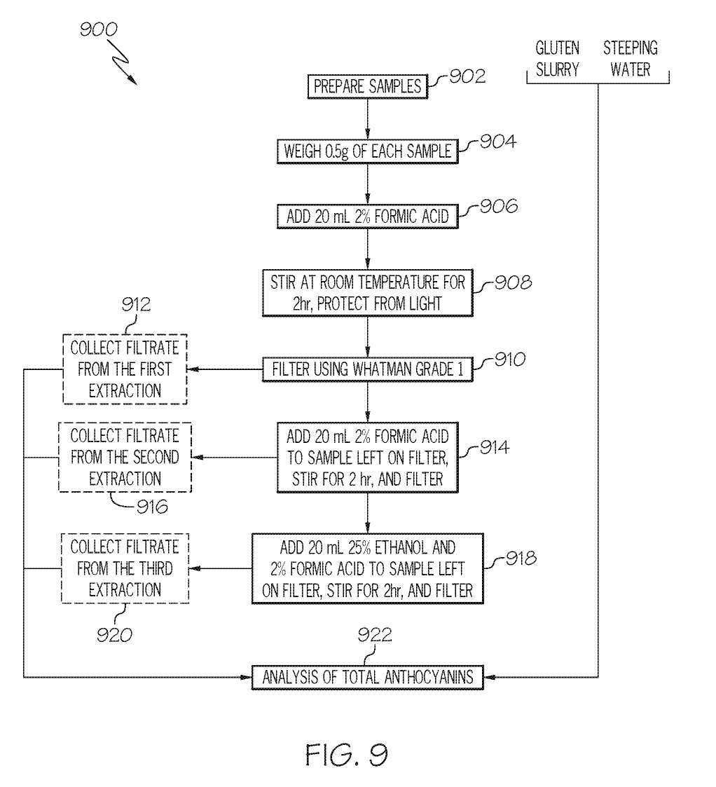

Whole corn kernels from the Maiz Morado corn cultivar were wet-milled to obtain solid samples. The procedure 900 of anthocyanin extraction from the wet-milled samples is schematically depicted in FIG. 9. Briefly, at step 902, the wet-milled solid samples were prepared. More particularly, the wet-mill solid samples, including germ, fiber, starch, and gluten, were ground using a Kitchen-Aid coffee grinder for 25 sec. The ground material was passed through a 35-mesh sieve and the material which did not initially pass through the sieve was ground again for another 25 sec and again passed through the 35-mesh sieve. The ground materials were combined and used for anthocyanin extraction.

Approximately 0.5 g of ground material was weighed at step 904 and was suspended in 20 mL (40:1 liquid-to-solid ratio) 2% v/v aqueous formic acid solution at step 906. The ground material and formic acid solution was mixed for 2 h at room temperature (22° C.) at step 908. The suspension was filtered using a Whatman grade 1 filter at step 910, and the filtrate was used for total monomeric anthocyanins (pH differential method) measurement (step 912). After the first extraction, samples remaining on the filter were mixed with 20 mL of 2% formic acid and again stirred at room temperature (22° C.) for 2 h for the second extraction. The suspension from the second extraction was also filtered (step 914) and the filtrate was collected (step 916). Solid samples remaining on the filter were mixed with 20 mL of 25% ethanol, 2% formic acid and stirred at room temperature (22° C.) for 2 h for the third extraction. The suspension from the third extraction was also filtered (step 918) and the filtrate was collected (step 920). Gluten slurry was filtered and the filtrate used for anthocyanin analysis at step 922.

(2) Pericarp Fiber Steeping

Corn pericarp fiber was prepared by the dry-milling procedure described in U.S. Pat. No. 6,254,914, the contents of which are incorporated herein by reference. Steeping treatments were as follows: 1. 10 g of pericarp fiber was mixed with 125 mL of deionized water (labeled as “water” in FIGS.); 2. 10 g of pericarp fiber was mixed with 125 mL deionized water and 0.27 g sodium metabisulfite (labeled as “water+SO₂” in FIGS.); 3. 10 g of pericarp fiber was mixed with 125 mL deionized water and 0.58 mL 85% lactic acid (labeled as “water+lactic acid.” in FIGS.); and 4. 10 g of pericarp fiber was mixed with 125 mL deionized water, 0.27 g sodium metabisulfite and 0.58 mL 85% lactic acid (labeled as “water+SO₂+lactic acid” in FIGS.). The four samples were incubated at 100 rpm at 52° C. for 24 h. The aqueous steeping solution was separated from the pericarp fiber using a ceramic crucible. The pericarp fiber was dried at room temperature for the measurement of anthocyanin concentration.

(3) Extraction of Anthocyanin From Corn Pericarp Fiber After Steeping

After pericarp fiber steeping, the remaining pericarp fiber was dried at room temperature and then ground using a Kitchen-Aid coffee grinder for 25 sec. The ground material was passed through a 35-mesh sieve. The material that did not initially pass through the sieve was ground again for another 25 sec and passed through the 35-mesh sieve. Materials that passed through the sieve were combined and used for extraction. Approximately 0.5 g of ground material was suspended in 20 mL (40:1 liquid-to-solid ratio) 2% aqueous formic acid and mixed for 2 h at room temperature. The suspension was filtered and the resulting filtrate used to determine total monomeric anthocyanins (pH differential method), and HPLC/MS-MS analysis. After the first extraction, pericarp fiber was mixed with 20 mL 2% formic acid again and stirred at room temperature for 2 h for a second extraction. The suspension from the second extraction was also filtered and the filtrate was collected for further measurements.

Measurement of Monomeric Anthocyanin Concentrations

The pericarp steeping samples were analyzed for total monomeric anthocyanin concentration by the pH differential method using a microplate reader method in three independent replicates as described in Lee, J.; Durst, R. W.; Wrolstad, R. E. Determination of total monomeric anthocyanin pigment content of fruit juices, beverages, natural colorants, and wines by the pH differential method: collaborative study. Journal of AOAC International 2005, 88, 1269-1278, the contents of which are incorporated herein by reference. Samples were diluted using two buffers (pH 1.0, 0.25 M KCl buffer and pH 4.5, 0.40 M sodium acetate buffer). Two hundred μL of diluted solution at each pH were transferred to a 96-well plate and the absorbance read at 520 nm and 700 nm using a Synergy 2 multiwell plate reader (Biotek, Winooski, Vt.). The total monomeric anthocyanin concentration was calculated as cyanidin-3-glucoside (C3G) equivalents per L as described below:

Total monomeric anthocyanins (mg/L)=(A*MW*D*1000)/(ε*PL*0.45)

Where: A=(A520-A700) at pH1.0-(A520-A700) at pH4.5; MW=449.2 g/mol for C3G; D=dilution factor; PL=constant path length 1 cm; ε=26900 L/mol-cm which is the molar extinction coefficient for C3G, 1000 is the conversion factor from grams to milligrams and 0.45 is the conversion factor from the established method to the plate reader method. Final results were then expressed as mg C3G equivalents per g fraction.

Measurement of Total Polyphenol Concentrations

Total polyphenol was measured using the Folin-Ciocalteu method adapted to a microassay as described in Heck, C I; Schmalko, M. de Mejía, E G. Effect of growing and drying conditions on the phenolic composition of mate teas (Ilex paraguariensis). Journal of Agricultural and Food Chemistry 2008, 56(18):8394-403, the entirety of which is incorporated herein by reference. Samples were diluted to a factor of 1:40 with deionized water. 50 μL of diluted samples, standard or blank (deionized water) were added to a 96-well plate, plus 50 μL of 1N Folin-Ciocalteu's phenol reagent. After 5 minutes, 100 μL of 20% Na₂CO₃ were added and the mixture allowed to stand for another 10 minutes. The absorbance was read at 690 nm using a Synergy multiwall plate reader (Biotek, Winooski, Vt.) and the results expressed as mg gallic acid/g pericarp fiber.

Measurement of Total Flavonoid Concentration

Total flavonoid concentration was measured using absorbance at 400 nm method as reported previously in Bower, A. M.; Hernandez, L. M.; Berhow, M. A.; Mejia, E. Bioactive compounds from culinary herbs inhibit a molecular target for type 2 diabetes management, dipeptidyl peptidase IV. Journal of Agricultural and Food Chemistry 2014, 62, 6147-6158, the entirety of which is incorporated herein by reference. Briefly, 50 μL of diluted samples, rutin standards or blank (methanol) were added to a 96-well plate. Then, 180 μL of methanol and 20 μL of 1% aminoethylborinate in methanol were added. The absorbance was read at 400 nm using a Synergy 2 multiwell plate (Biotek, Winooski, Vt.). Results were expressed as μg rutin per gram of pericarp fiber.

Measurement of Total Tannin Concentrations

The method used to measure total tannins was based on the method reported previously in Mojica, L; Meyer, A; Berhow, M A; Gonzalez de Mejía, E. Bean cultivars (Phaseolus vulgaris L.) have similar high antioxidant capacity, in vitro inhibition of α-amylase and α-glucosidase while diverse phenolic composition and concentration. Food Research International 2015, 69, 38-48, the entirety of which is incorporated herein by reference. Briefly, a 50 μL of diluted sample (methanol) and catechin standard or blank were added to each well followed by the addition of 200 μL of 8% acidified methanol and 50 μL of 1% vanillin (1:1) mixture until completing 200 μL. Fifty μL of methanol and 200 μL of 4% acidified methanol were used as blank. Absorbance was read at 500 nm, with filter from 492 to 540 nm using a Synergy 2 multiwell plate reader (BioTek, Winooski, Vt.). The amount of condensed tannins was calculated and expressed as mg catechin equivalents per gram of pericarp fiber.

HPLC/MS-MS Analysis

HPLC analysis of samples for anthocyanin profile was performed in triplicate using a Hitachi HPLC System (Hitachi High Technologies America, Inc., Schaumburg Ill.) equipped with a multi-wavelength detector, L-7100 pump following previously reported protocol (West and Mauer, 2013) with some modifications. The flow rate was 1 mL/min and the gradient used was from 2% formic acid in water and 0% acetonitrile to 40% acetonitrile in a linear fashion using a Grace Prevail C18 (5 μm, 250×4.6 mm, Columbia, Md.) for 30 min. The injection volume was 20 μL with a flow rate of 1 mL/min. For MS/MS analysis, an electrospray ionization-time-of-flight system (Bruker Daltoniks, Billerica, Mass.) was used with the following conditions: Survey ion mode, ES mode; polarity, positive; scan range, 50-950 m/z; source temperature, 114° C.; desolvation temperature, 248° C.; cone gas flow, 964 L/h; and desolvation gas flow, 1175 L/h.

Analysis

All analyses were conducted in at least three independent replicates. SAS version 9.4 software was used; statistical differences among independent variables were determined using ANOVA by the proc GLM procedure and LSD posthoc test (p<0.05). Correlations were also performed using Office Excel.

The data collected demonstrate that the aqueous steeping solution from the wet-milled samples contained the majority of the anthocyanins extracted from the purple corn variety. In order to study the distribution of anthocyanins in products manufactured by wet-milling, the concentrations of total extractable anthocyanin in each wet-milling co-product, including germ, fiber, starch, gluten slurry, and aqueous steeping solution were measured. The results are reported in Table 1, below. Three sequential extractions of anthocyanins from solid samples were conducted as described above, in triplicate. Anthocyanins from the first extraction of the fiber were not significantly different from the second or third extractions, which contained 26.8, 15.5, and 24.9 mg C3G equivalent/kg of whole corn, respectively, for a total of 67.2±2.9 mg C3G equivalent/kg. This suggests that anthocyanins are closely attached to the fiber, which decreased the extraction rate. However, the first extraction of anthocyanins from the germ, starch, and gluten were all significantly higher than the second extraction, as shown in Table 1. Fiber, germ, starch, gluten, gluten slurry, and aqueous steeping solution contributed to 1.36%, 0.69%, 1.59%, 6.25%, 10.64%, and 79.12% of the total extractable anthocyanins from whole corn kernel, respectively. Because the aqueous steeping solution contained a significant amount of anthocyanins of the whole corn kernel, the chemical characteristics of the aqueous steeping solution extracts from purple corn pericarp fiber were investigated further after isolating the pericarp fiber from the remainder of the constituent component parts of the corn kernel, as described above.

TABLE 1
Total monomeric anthocyanins of purple Maiz Morado whole
corn and co-products from the wet-milling process

| | | | | | | Relative |
|---|---|---|---|---|---|---|
| | | Anthocyanin (mg/kg of whole corn)^{#} | | | | anthocyanin |
| | Fraction/ | | | | Sum of | (Co- |
| | co- | First | Second | Third* | sequential | Product/Whole |
| Method | product ^{&} | extraction | extraction | extraction | extractions | Corn) |
| Wet- | Fiber | 26.8 ± 9.4a | 15.5 ± 7.4a | 24.9 ± 3.5a | 67.2 ± 2.9 | 1.36 |
| milling | | | | | | |
| | Germ | 22.5 ± 5.3a | 6.2 ± 0.7b | 5.3 ± 2.4b | 34.0 ± 5.5 | 0.69 |
| | Starch | 69.9 ± 15.9a | 8.5 ± 3.1b | 0.0b | 78.4 ± 14.5 | 1.59 |
| | Gluten | 114.7 ± 19.7a | 84.5 ± 10.8b | 109.0 ± 8.6ab | 308.2 ± 14.1 | 6.25 |
| | Gluten | | | | 525.0 ± 6.4 | 10.64 |
| | slurry | | | | | |
| | Aqueous | | | | 3903.0 ± 58.2 | 79.12 |
| | steeping | | | | | |
| | solution | | | | | |
| Whole | | | | | 4933.1 ± 43.4 | 100 |
| corn | | | | | | |

^{&} Two independent batches of wet-milling samples were tested.
*For the third extraction, sample remaining on filter was added with 20 mL 25% ethanol, 2% formic acid, stir for 2 h, and filtered.
^{#}Values represent means ± SEM of three individual extractions of each sample.

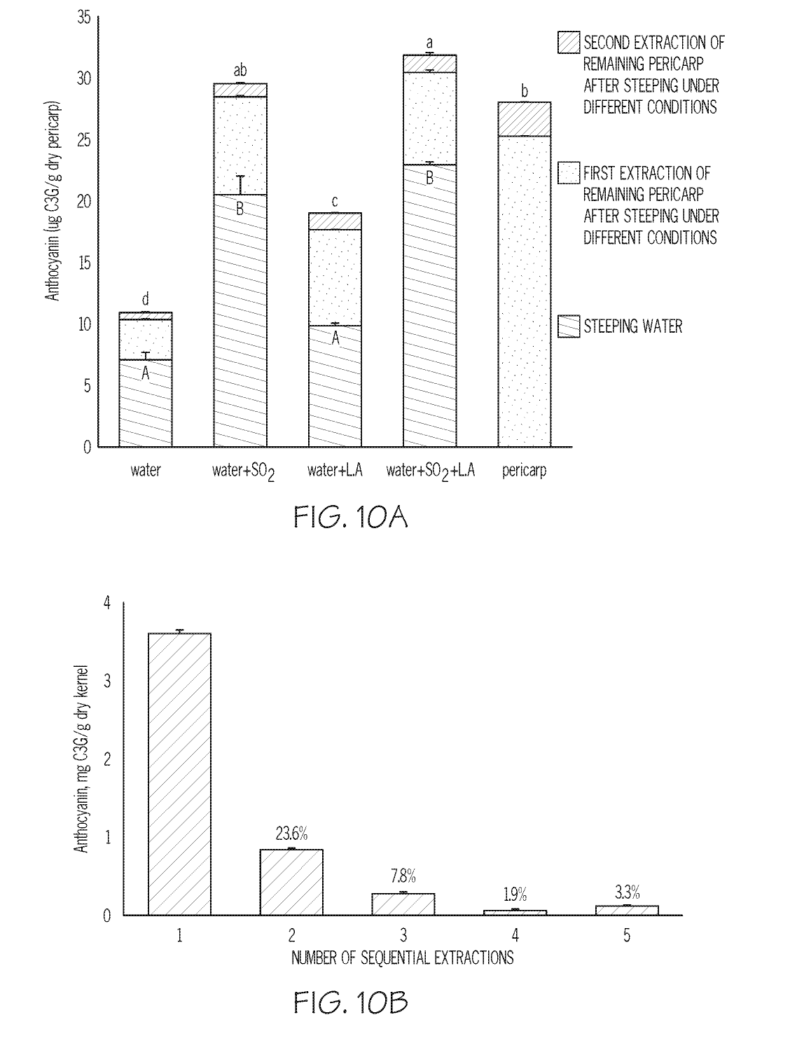

FIG. 10A shows the anthocyanin concentration in the aqueous steeping solution (water only), aqueous steeping solution with SO₂, aqueous steeping solution with lactic acid, and aqueous steeping solution with both SO₂ and lactic acid. The data shows that samples processed with SO₂ and SO₂+lactic acid had the highest anthocyanin concentration, which were equivalent to 20.5±1.5 mg and 22.95±0.24 mg C3G equivalent/g dry pericarp, respectively. Total extractable anthocyanin concentration in the aqueous steeping solution with lactic acid was not different (p>0.05) from the aqueous steeping solution, indicating that lactic acid itself did not increase the extraction of anthocyanins from the pericarp fiber.

The extracted anthocyanins from the remaining dried pericarp fiber using 2% formic acid showed that there was still a large amount of anthocyanin remained in the pericarp after the aqueous steeping solution extraction. During the first extraction from the pericarp with water, water+SO₂, water+lactic acid, or water+SO₂+lactic acid, the concentration of anthocyanins increased by 3.3±0.6, 8.0±1.31, 7.8±0.4, and 7.49±0.9 mg C3G equivalent/g dry pericarp fiber, respectively. The second extraction yielded much less anthocyanin than the first extraction, which were 0.6±0.07, 1.1±0.09, 1.4±0.03, and 1.4±0.2 mg equivalent/g dry pericarp fiber, respectively. An extraction of anthocyanins directly from the solid pericarp fiber generated from a corn dry-milling process, using 2% formic acid, revealed a higher concentration of 22.5 mg C3G equivalent/g of dry pericarp fiber. Total extractable anthocyanin concentration extracted from the aqueous steeping solution and remaining pericarp in water, water+SO₂, water+lactic acid, water+SO₂+lactic acid, and pericarp fiber directly from dry-milling were 10.9±0.8, 29.5±0.2, 19.0±0.3, 31.9±0.9, and 28.0±0.09 mg C3G equivalent/g of pericarp, respectively. Total extractable anthocyanins in SO₂+lactic acid products were even higher than that directly extracted with formic acid from pericarp fiber (p<0.05), suggesting that SO₂ assisted with lactic acid significantly enhanced the anthocyanin extraction from the pericarp fiber.

In order to estimate the yield of extractable anthocyanins directly from corn kernels (rather than only the pericarp fiber of the corn kernels), 2% formic acid was used to extract anthocyanins from ground purple corn kernels. The first extraction achieved 3.6±0.03 mg C3G/g kernel. The second extraction contributed to 23.6% anthocyanin of the first extraction and the third and fourth extractions also withdrew 7.8% and 1.9% of the first extraction, respectively. When 20% ethanol was used for the fifth extraction, it resulted in an additional 3.3% anthocyanin of the first extraction, as depicted in FIG. 10B. Total extractable anthocyanin concentration from the five extractions yielded a total 4.9±0.07 mg C3G/g kernel.

It was also determined that the total polyphenols, flavonoids, and tannins concentrations of the pericarp fiber aqueous steeping solution co-products were affected by the type of treatment used to extract the anthocyanins from the pericarp fiber. Total polyphenol concentration in water+SO₂ was significantly higher than in water+lactic acid, which were 47.1±2.6 and 28.3±0.3 mg gallic acid equivalent/g dry pericarp, respectively (p<0.05), as shown in FIG. 11A. Even though total polyphenols in water+SO₂ were not significantly different from water or water+SO₂+lactic acid groups (p>0.05), the average of polyphenol concentration in water+SO₂ was higher than the other two groups. Total polyphenol concentration in formic acid extracts from pericarp were 53.0±0.03 mg gallic acid equivalent/g dry pericarp fiber, which was significantly higher than those extracted with water and water+lactic acid group (p<0.05), but not different from water+SO₂ or water+SO₂+lactic acid (p>0.05).

Total flavonoids concentrations among pericarp fiber aqueous steeping solution co-products were not different from one another. However, the flavonoid concentrations in pericarp fiber after extraction with formic acid was 23.0±0.52 mg rutin equivalent/g dry pericarp; 100% more than the steeping liquids, as shown in FIG. 11B.

In general, tannin concentrations among aqueous steeping solution co-products revealed similar patterns to total polyphenols. Water+SO₂, and water+SO₂+lactic acid tannin concentrations were significantly higher than water and water+lactic acid, as depicted in FIG. 11C, indicating that SO₂ played an important role in potential de-polymerization of tannins as well. Again, formic acid extracts from the pericarp fiber showed the highest total tannin concentration, which was 1092.9±22.6 mg catechin equivalent/g dry pericarp fiber (p<0.05).

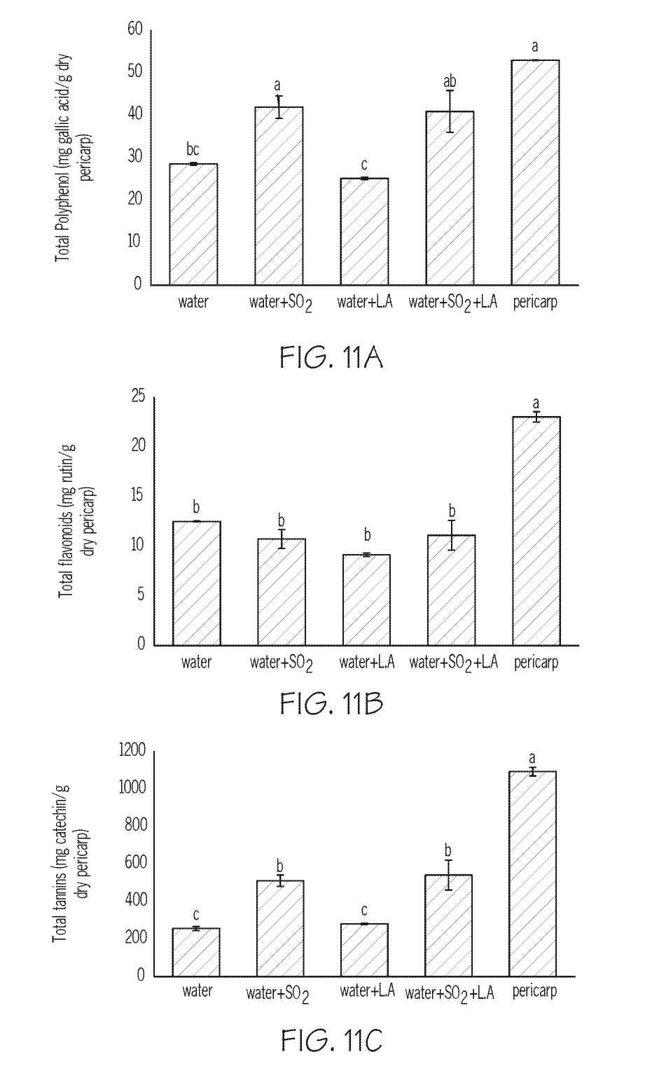

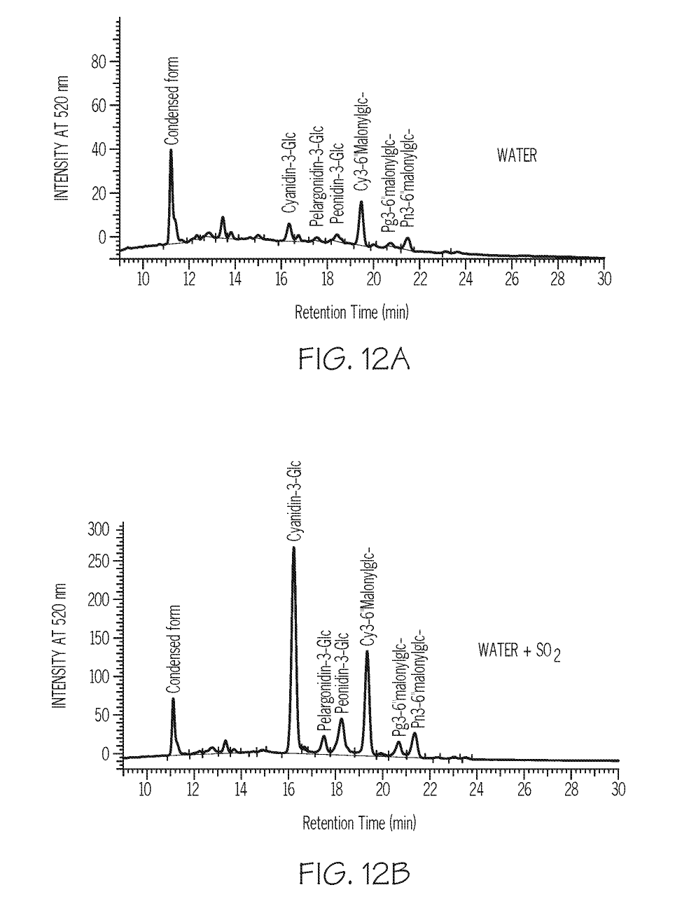

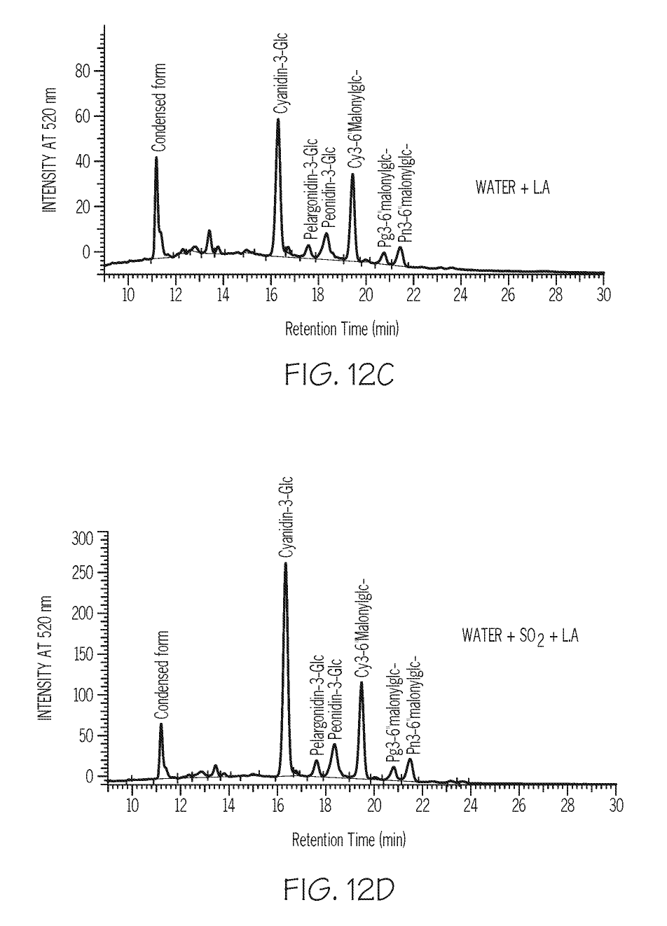

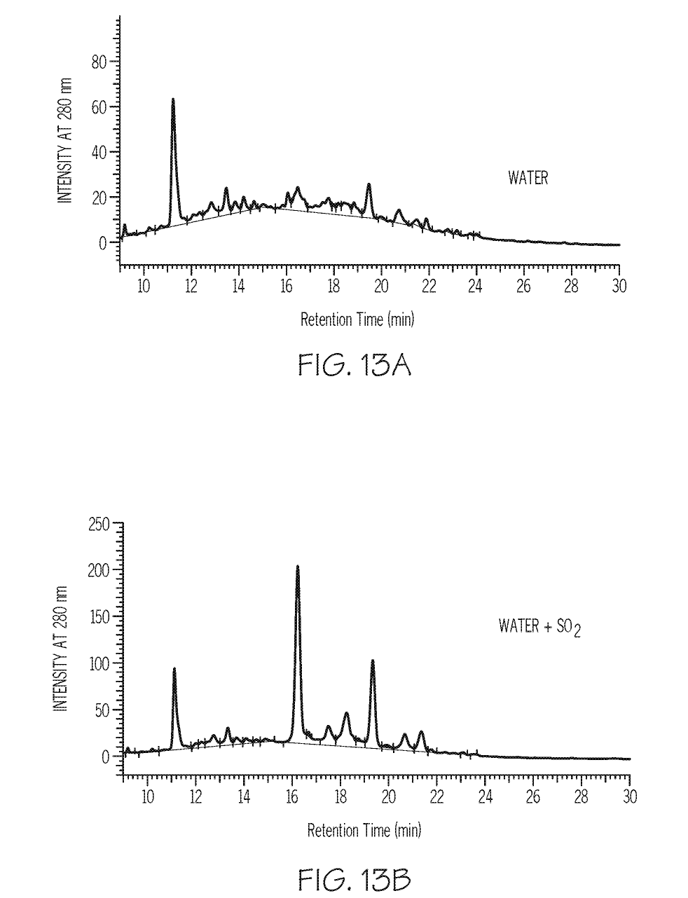

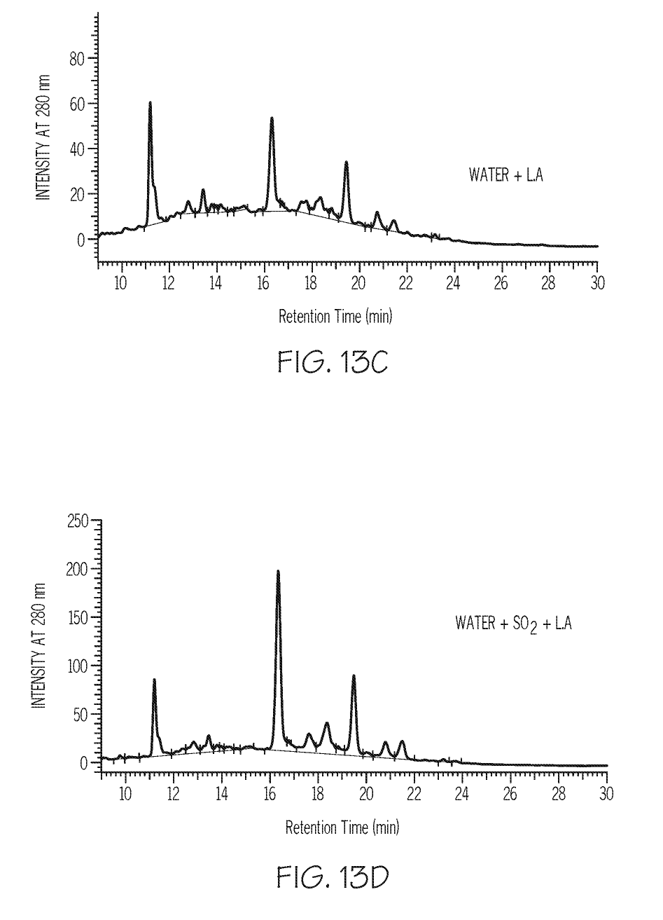

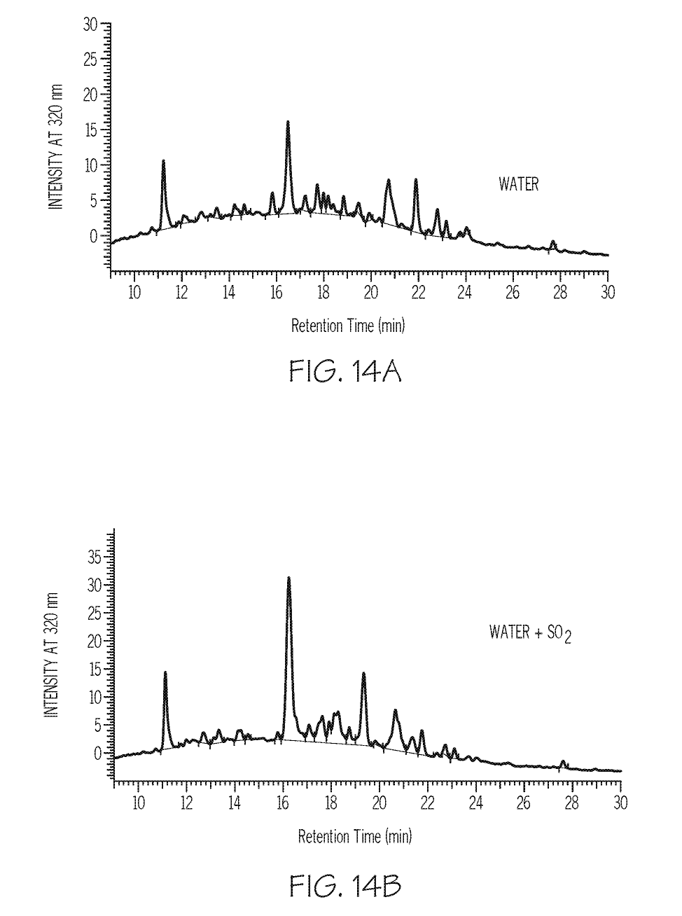

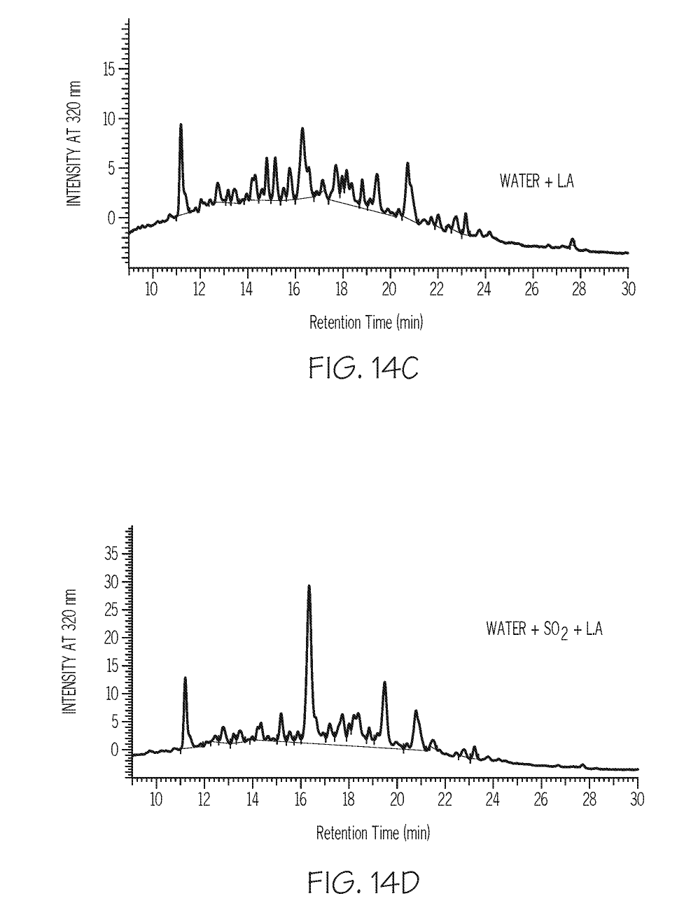

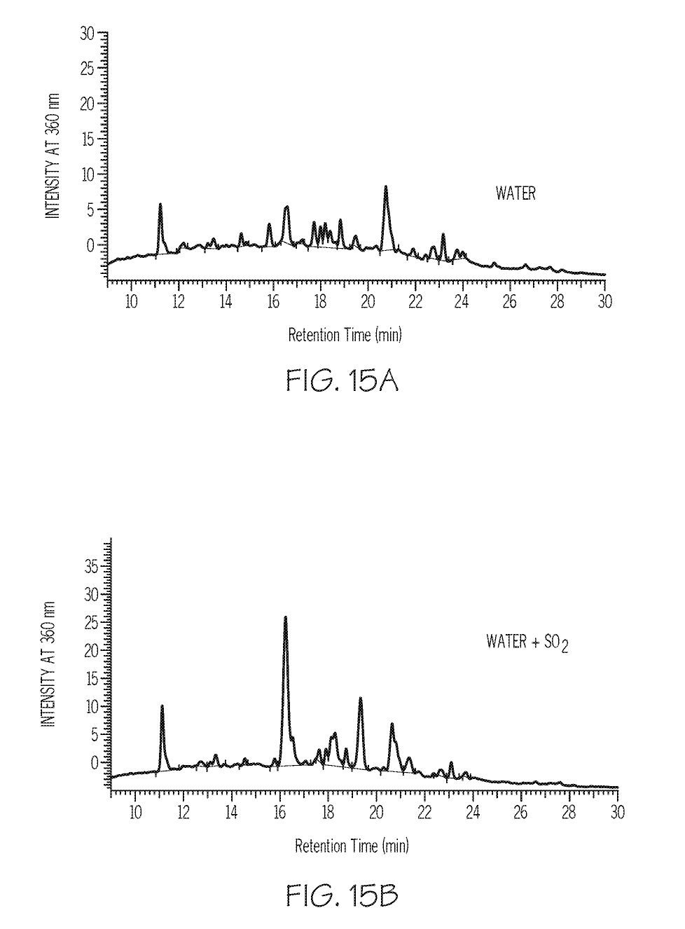

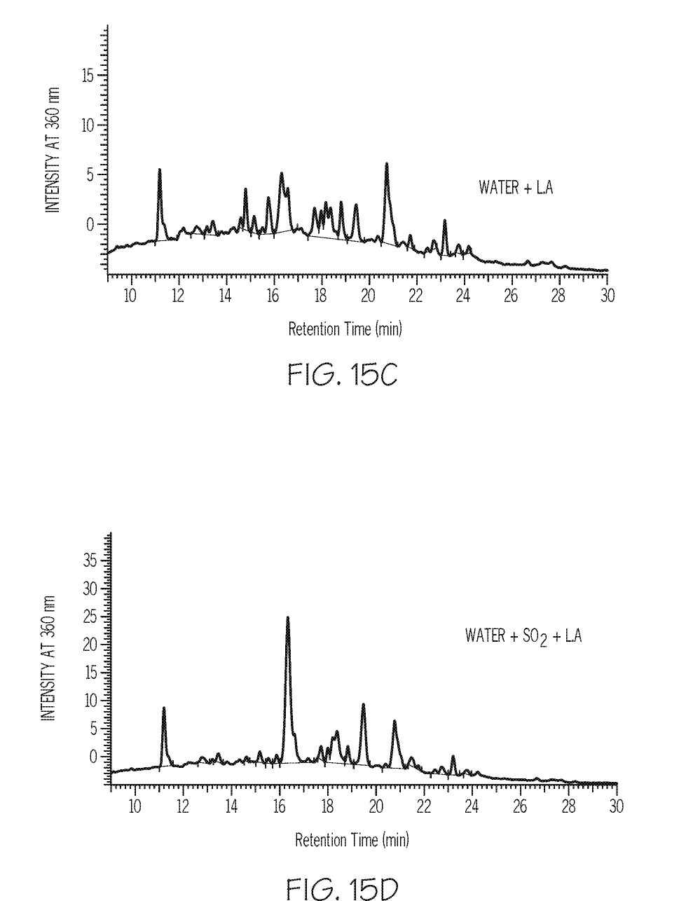

It was also determined that the type of anthocyanin in the aqueous steeping solution co-products varied with the extraction treatment. In the aqueous steeping solution, the predominant component was the condensed form (37%), as shown in FIG. 12A. But in all the other three treatments, i.e. SO₂, lactic acid, SO₂+lactic acid, the dominant anthocyanin was C3G, as shown in FIGS. 12B, 12C, and 12D, respectively. HPLC results at 280 nm (FIG. 13), 320 nm (FIG. 14), and 360 nm (FIG. 15) detected phenolics, conjugated forms of hydroxycinamic acids, and flavonols, respectively. Proanthocyanidins were highly present in the aqueous steeping solution co-products, but reduced by SO₂, as indicated in FIG. 13. Mass spectrometry identified possible structures of major peaks shown by HPLC as indicated in Table 2.

TABLE 2
Identification of possible anthocyanins in pericarp aqueous steeping
solution co-products from purple corn by HPLC-Mass Spectrometry analyses

| | Rt (min) | M+ | MS/MS fragments | Possible identity^{a} |
|---|---|---|---|---|
| Water | 17.83 | 899 | 575, 737, 423, 329, 287 | catechin-(4,8)-cyanidin-3,5-diglucoside |
| | 27.07 | 449 | 287, 288 | Cyanindin-3-glucoside |
| | 34.19 | 535 | 287, 288 | cyanidin-3-(6″-malonyl)glucoside |
| Water + | 17.83 | 899 | 575, 737, 423, 329, 287 | catechin-(4,8)-cyanidin-3,5-diglucoside |
| SO₂ | 26.74 | 449 | 287, 288 | Cyanindin-3-glucoside |
| | 31.00 | 463 | 301 | Peonidin-3-glucoside |
| | 34.00 | 535 | 287, 288 | cyanidin-3-(6″-malonyl)glucoside |
| | 37.80 | 549 | 301 | peonidin-3-(6″-malonyl)glucoside |
| Water + | 17.90 | 899 | 575, 737, 423, 329, 287 | catechin-(4,8)-cyanidin-3,5-diglucoside |
| lactic acid | 27.24 | 449 | 287, 288 | Cyanindin-3-glucoside |
| | 31.00 | 463 | 301 | Peonidin-3-glucoside |
| | 34.77 | 535 | 287, 288 | cyanidin-3-(6″-malonyl)glucoside |
| | 37.50 | 549 | 301 | peonidin-3-(6″-malonyl)glucoside |

^{a}The identification of major peaks were referred in Dia, VP; Wang, Z; West, M; Singh, V; West, L; Gonzalez de Mejia, E. Processing Method and Corn Cultivar Affected Anthocyanin Concentration from Dried Distillers Grains with Solubles. Journal of Agricultural and Food Chemistry 2015, 63 (12), 3205-3218.

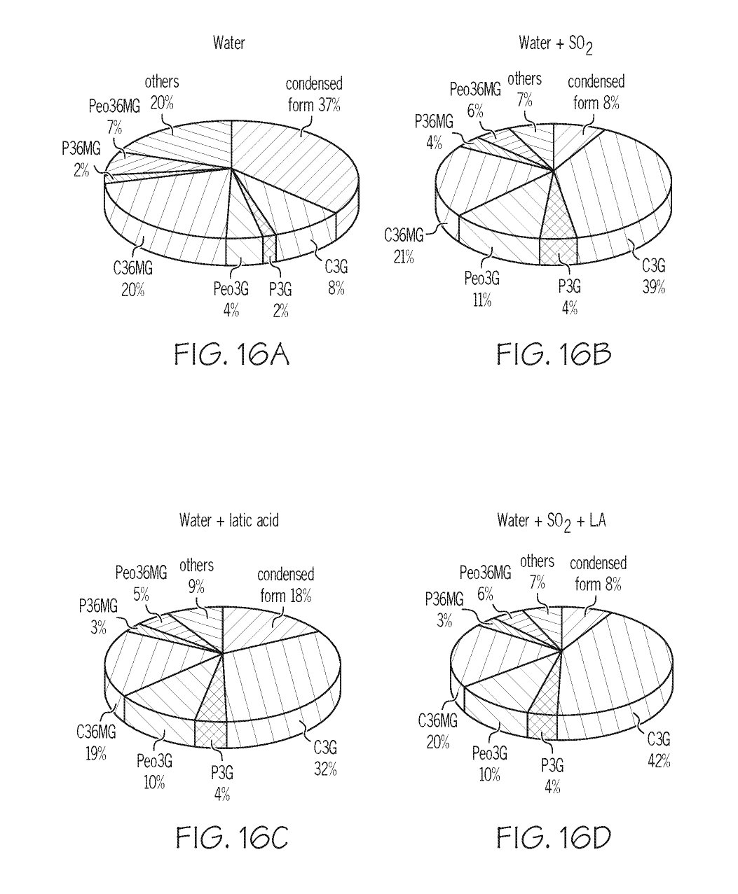

FIG. 16 presents the percentage (%) of anthocyanin distribution in pericarp aqueous steeping solution co-products, only water (A), water+SO₂ (B), water+lactic acid (C), and water+SO₂+lactic acid (D).

The data in Table 1 show that aqueous steeping solution contributed 88.4% of total extractable anthocyanin from Maiz Morado purple corn. The extraction of anthocyanins from pericarp fiber versus whole corn kernels resulted in 28 mg and 4.9 mg C3G/g product, respectively. Because 1 kg of corn generated 80 g pericarp fiber, therefore, the pericarp fiber contributed to 44% of anthocyanins from the whole corn kernel. Concentration of sulfur salts resulting in 2000 ppm SO₂, produced maximum yields of total phenolics and anthocyanins. The data also show that the presence of SO₂ in the aqueous steeping solution enhanced total extractable anthocyanin extraction by 200%. While not wishing to be bound by theory, possible reasons for the high extraction of anthocyanins by SO₂ are interactions of anthocyanins with HSO₃⁻ ions leading to improved diffusion through cell walls and increased solubility of the pigments. HPLC at 280 nm detected the presence of phenolic compounds. A peak in HPLC at 280 nm is usually associated with the presence of proanthocyanidins, in this case in both water and water+lactic acid groups, as depicted in FIG. 13. However, no peak was present in water+SO₂ or water+SO₂+lactic acid products, suggesting SO₂ may play a role in the depolymerization of proanthocyanidin to small basic units, which results in the increase of anthocynins.

The aqueous steeping solution containing SO₂ contained total extractable anthocyanin equivalents of 20.5±1.5 mg cyanidin-3-glucoside (C3G)/g dry pericarp fiber, which was significantly higher than aqueous steeping solutions with only water (7.1±0.6 mg C3G/g dry pericarp, p<0.05). Lactic acid did not change total extractable anthocyanin concentrations in the first extraction; however two further extractions yielded significantly higher concentrations. The combination of SO₂ and lactic acid significantly increased total extractable anthocyanin concentrations up to 22.9±0.2 mg C3G/g dry pericarp (p<0.05). This was associated with increased C3G and decreased condensed forms of anthocyanins as compared to an aqueous steeping solution only using water. Chroma was highly correlated to the concentration of anthocyanins (r=0.999) and tannins (r=0.994).

### Example 2

A study was conducted to assess the effect of different types of milling on total extractable anthocyanin extraction from the pericarp fiber of purple corn. Corn kernel pericarp fiber was prepared by dry-milling whole corn kernels. The pericarp fiber was then ground either by ball milling or hammer milling to produce anthocyanin-containing cellular material fractions and non-anthocyanin containing cellular material fractions. The milled pericarp fiber was then sieved using 1 of 5 different sieve sizes (40, 60, 80, 120, or fine sieve number) to separate the anthocyanin-containing cellular material fractions from the non-anthocyanin containing cellular material fractions. The recovered anthocyanin-containing cellular material fraction was then chemically processed with formic acid to extract the anthocyanins therefrom in three consecutive extractions.

Specifically, the samples of the separated ground pericarp fiber were suspended in 20 mL (40:1 liquid-to-solid ratio) 2% v/v aqueous formic acid solution and mixed for 2 h at room temperature (22° C.). The suspension was filtered and the filtrate was used for total monomeric anthocyanins (pH differential method) measurement. After the first extraction, samples remaining on the filter were mixed with 20 mL of 2% formic acid and again stirred at room temperature (22° C.) for 2 h for the second extraction. The suspension from the second extraction was also filtered and the filtrate was collected. Solid samples remaining on the filter were mixed with 20 mL of 25% ethanol, 2% formic acid and stirred at room temperature (22° C.) for 2 h for the third extraction. The suspension from the third extraction was also filtered and the filtrate was collected.

Table 4 below shows the total extractable anthocyanin (mg C3G equivalent/g pericarp fiber) recovered from each extraction of the ball milled samples. For the first formic acid extraction, total extractable anthocyanin of pericarp fiber sieved through the No. 120 sieve was significantly higher than the pericarp fiber sieved through the No. 40 sieve. There was not a significant difference between the anthocyanins recovered from the material sieved through the No. 120 sieve and the No. 60 and No. 80 sieves. For the sum of the three sequential extractions, anthocyanin extractions from the pericarp fiber sieved by No. 120 sieve was significantly higher than both No. 40 sieve and the fines sieve. Again, there was not a significant difference between the material sieved through the No. 120, No. 60, and No. 80 sieves. This data indicates that the smaller sieve sizes (No. 120, No. 80, and No. 60) were more effective in isolating the anthocyanin-containing cellular material from the non-anthocyanin cellular material down to a size of 125 microns, thereby increasing the yield of the anthocyanin from the pericarp fiber and the efficiency of the process.

TABLE 4
Total extractable anthocyanin (mg C3G equivalent/g pericarp)

| | | | | | Sum |
|---|---|---|---|---|---|
| | | | | | of three |
| Sieve | Size | First | Second | Third | sequential |
| number | of sieve | extraction | extraction | extraction* | extractions |
| 40 | 425 μm | 28.5 ± | 7.1 ± | 3.6 ± | 39.2 ± |
| | | 1.5 b | 0.4 a | 0.2 a | 1.0 b |
| 60 | 250 μm | 37.0 ± | 7.4 ± | 3.9 ± | 48.3 ± |
| | | 2.6 ab | 1.5 a | 0.3 a | 3.7 ab |
| 80 | 180 μm | 34.3 ± | 8.5 ± | 4.7 ± | 47.5 ± |
| | | 2.7 ab | 0.3 a | 0.2 a | 2.4 ab |
| 120 | 125 μm | 38.5 ± | 9.0 ± | 4.3 ± | 51.9 ± |
| | | 0.8 a | 0.1 a | 0.5 a | 1.1 a |
| Fines | <125 μm | 30.0 ± | 7.1 ± | 3.5 ± | 40.6 ± |
| | | 0.3 ab | 0.1 a | 0.1 a | 0.3 b |

*For the third extraction, samples left on filter was added with 20 mL 25% ethanol, 2% formic acid, stir for 2 h, and filtered. These values represent means ± SEM of three individual extractions of each sample. Different letters behind the values represent significant difference among 5 different sieve numbers (p < 0.05).

Table 5 below shows the total extractable anthocyanin (mg C3G equivalent/g pericarp fiber) recovered from each extraction of the hammer milled samples. For the first formic acid extraction, total extractable anthocyanin of pericarp fiber sieved through the No. 80 sieve was significantly higher than the pericarp fiber sieved through the No. 40 sieve, the No. 120 sieve, and the fines sieve. There was not a significant difference between the anthocyanins recovered from the material sieved through the No. 80 sieve and the No. sieve. For the sum of the three sequential extractions, anthocyanin extractions from the pericarp fiber sieved by No. 80 sieve was significantly higher than both No. 40 sieve and the fines sieve. There was not a significant difference between the material sieved through the No. 120, No. 60, and No. 80 sieves. This data indicates that the smaller sieve sizes (No. 120, No. 80, and No. 60) were more effective in isolating the anthocyanin-containing cellular material from the non-anthocyanin cellular material down to a size of 125 microns, thereby increasing the yield of the anthocyanin from the pericarp fiber and the efficiency of the process.

TABLE 5
Total extractable anthocyanin (mg C3G equivalent/g pericarp)

| | | | | | Sum of |
|---|---|---|---|---|---|
| Sieve | | | | | three |
| Num- | Sieve | First | Second | Third | sequential |
| ber | Size | extraction | extraction | extraction* | extractions |
| 40 | 425 | 19.4 ± 2.6 cd | 6.5 ± 1.2 a | 2.3 ± 0.3 c | 28.2 ± 4.1 b |
| | μm | | | | |
| 60 | 250 | 28.2 ± 1.1 ab | 7.5 ± 0.2 a | 3.5 ± 0.3 ab | 39.3 ± 1.0 a |
| | μm | | | | |
| 80 | 180 | 33.9 ± 0.5 a | 8.5 ± 0.7 a | 4.5 ± 0.2 a | 46.8 ± 0.9 a |
| | μm | | | | |
| 120 | 125 | 26.0 ± 1.6 bc | 6.4 ± 0.8 a | 4.0 ± 0.1 ab | 36.4 ± 2.4 ab |
| | μm | | | | |
| Fines | <125 | 18.2 ± 0.8 d | 5.5 ± 0.2 a | 3.0 ± 0.1 bc | 26.7 ± 1.1 b |
| | μm | | | | |

*For the third extraction, sample left on filter was added with 20 mL 25% ethanol, 2% formic acid, stir for 2 h, and filtered. These values represent means ± SEM of three individual extractions of each sample. Different letters behind the values represent significant difference among 5 different sieve numbers (p < 0.05).

A comparison of the data in Tables 4 and 5 indicates that the extraction data from ball milling and hammer milling generally correlates with respect to greater anthocyanin concentrations being extracted from material sieved through the Nos. 60, 80, and 120 sieves than the Nos. 40 and fines sieves. The data also indicates that the ball milling process generally yielded greater concentrations of anthocyanins after extraction than the hammer milling process.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A method of extracting anthocyanins from corn kernels, the method comprising:
fractionating the corn kernels into their constituent component parts;
separating pericarp fiber from the constituent component parts of the corn kernels; and
steeping the pericarp fiber in an aqueous solution to extract anthocyanins from the pericarp fiber, wherein, after steeping, the aqueous solution contains greater than about 40% by weight of total extractable anthocyanins present in the corn kernels prior to fractionating.

2. The method of claim 1, wherein fractionating the corn kernels comprises wet milling the corn kernels.

3. The method of claim 1, wherein fractionating the corn kernels comprises dry milling the corn kernels.

4. The method of claim 1, wherein fractionating the corn kernels comprises dry grinding the corn kernels.

5. The method of claim 1, wherein the aqueous solution comprises deionized water.

6. The method of claim 1, wherein the aqueous solution comprises water and at least one reducing compound.

7. The method of claim 6, wherein the at least one reducing compound is a sulfite compound.

8. The method of claim 6, wherein the at least one reducing compound is present in the aqueous solution in a concentration from about 5 parts per million to about 4000 parts per million.

9. The method of claim 6, wherein the at least one reducing compound is present in the aqueous solution at a concentration from about 1500 parts per million to about 2000 parts per million.

10. The method of claim 6, wherein the aqueous solution further comprises at least one organic acid.

11. The method of claim 10, wherein the at least one organic acid comprises at least one of acetic acid, formic acid, propionic acid, butyric acid, valeric acid, caproic acid, oxalic acid, lactic acid, malic acid, citric acid, benzoic acid, and carbonic acid.

12. The method of claim 10, wherein the at least one organic acid is present in the aqueous solution in a concentration from about 0.01% by weight to about 20% by weight of the aqueous solution.

13. The method of claim 10, wherein the at least one organic acid is present in the aqueous solution in a concentration from about 0.5% by weight to about 2% by weight of the aqueous solution.

14. The method of claim 1, wherein the aqueous solution comprises water and at least one organic acid.

15. The method of claim 14, wherein the at least one organic acid comprises at least one of acetic acid, formic acid, propionic acid, butyric acid, valeric acid, caproic acid, oxalic acid, lactic acid, malic acid, citric acid, benzoic acid, and carbonic acid.

16. The method of claim 14, wherein the at least one organic acid is present in the aqueous solution in a concentration from about 0.01% by weight to about 20% by weight of the aqueous solution.

17. The method of claim 14, wherein the at least one organic acid is present in the aqueous solution in a concentration from about 0.5% by weight to about 2% by weight of the aqueous solution.

18. The method of claim 1, wherein the aqueous solution comprises water, at least one sulfite compound, and lactic acid.

19. The method of claim 1, further comprising grinding the pericarp fiber after separating the pericarp fiber from the corn kernels to obtain ground pericarp.

20. The method of claim 19, further comprising sieving the ground pericarp after grinding the pericarp fiber to separate anthocyanin-containing cellular material from non-anthocyanin-containing cellular material, wherein the anthocyanin-containing cellular material is steeped in the aqueous solution.

21. The method of claim 20, further comprising separating the anthocyanin-containing cellular material from the non-anthocyanin-containing cellular material based on density, wherein the anthocyanin-containing cellular material is steeped in the aqueous solution.

22. The method of claim 21, wherein the anthocyanin-containing cellular material is separated from the non-anthocyanin-containing cellular material by float separating the ground pericarp.

23. The method of claim 1, wherein steeping the pericarp fiber comprises steeping the pericarp fiber in the aqueous solution for about 10 minutes to about 48 hours.

24. The method of claim 1, wherein steeping the pericarp fiber comprises steeping the pericarp fiber in the aqueous solution at a temperature from about 20° C. to about 95° C.

25. The method of claim 1, wherein, after steeping, the aqueous solution contains greater than about 65% by weight of the total extractable anthocyanins present in the corn kernels prior to fractionating.

26. The method of claim 1, wherein, after steeping, the aqueous solution contains greater than about 67% by weight of the total extractable anthocyanins present in the corn kernels prior to fractionating.

27. The method of claim 1, wherein, after steeping, the aqueous solution contains greater than about 70% by weight of the total extractable anthocyanins present in the corn kernels prior to fractionating.

28. The method of claim 1, wherein:
fractionating the corn kernels comprises fractionating whole corn kernels with a degermination mill into a first fraction comprising grits and a second fraction comprising germ, pericarp fiber, and ground corn;
the second fraction is ground in a roller mill to obtain a ground second fraction; and
separating the pericarp fiber from the constituent component parts comprises passing the ground second fraction through a sieve to separate the pericarp fiber from the germ.

29. The method of claim 28, wherein steeping the pericarp fiber in the aqueous solution comprises combining the pericarp fiber with the aqueous solution in a ratio of mass of dry pericarp fiber (grams) to volume of aqueous solution (ml) from about 1:10 to about 1:100.

30. The method of claim 28, further comprising isolating the anthocyanins from the aqueous solution.

31. The method of claim 30, wherein isolating the anthocyanins from the aqueous solution and the pericarp fiber comprises:
removing the pericarp fiber from the aqueous solution, wherein the anthocyanins are contained in the aqueous solution;
combining the aqueous solution containing the anthocyanins with an extraction solution comprising at least one of an alcohol, an organic acid, and an enzyme; and
passing the aqueous solution, the anthocyanins, and the extraction solution through a filter to isolate the anthocyanins.

32. The method of claim 28, further comprising processing the first fraction to obtain one or more supplements, cosmeceuticals, cosmetics, industrial products, food products for human consumption, and non-human animal feed.
